SEQUENCE LISTING (41 sequences)

SEQ ID NO: 1 - DNA, 750 bp, Artificial
  nucleotide sequence encoding the human APRIL R206E full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggacgaggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 2 - protein, 250 aa, Artificial
  amino acid sequence of the human APRIL R206E full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDEAYNSCYSAGVFHLHQGDILSVIIPRARAKLNLSPHGTFLGFVKL

SEQ ID NO: 3 - DNA, 750 bp, Artificial
  nucleotide sequence encoding the human APRIL D132F full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactcctttgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 4 - protein, 250 aa, Artificial
  amino acid sequence of the human APRIL D132F full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSFVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 5 - DNA, 750 bp, Artificial
  nucleotide sequence encoding the human APRIL D132Y full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactcctatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 6 - protein, 250 aa, Artificial
  amino acid sequence of the human APRIL D132Y full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSYVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 7 - DNA, 750 bp, Artificial
  nucleotide sequence encoding the human APRIL R206M full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggacatggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 8 - protein, 250 aa, Artificial
  amino acid sequence of the human APRIL R206M full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDMAYNSCYSAGVFHLHQGDILSVIIPRARAKLNLSPHGTFLGFVKL

SEQ ID NO: 9 - DNA, 750 bp, Artificial
  nucleotide sequence encoding the human APRIL D205Y full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccgtatcgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 10 - protein, 250 aa, Artificial
  amino acid sequence of the human APRIL D205Y full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPYRAYNSCYSAGVFHLHQGDILSVIIPRARAKLNLSPHGTFLGFVKL

SEQ ID NO: 11 - DNA, 750 bp, Artificial
  nucleotide sequence encoding the human APRIL T175D full length variant
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtggatttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 12 - protein, 250 aa, Artificial
  amino acid sequence of the human APRIL T175D full length variant
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVDFTMGQVVSREGQGRQETLFRCIRSMPSHPDRAYNSCYSAGVFHLHQGDILSVIIPRARAKLNLSPHGTFLGFVKL

SEQ ID NO: 13 - DNA, 750 bp, Artificial
  nucleotide sequence encoding the human wild type APRIL full length protein
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 14 - protein, 250 aa, Artificial [sequence omitted]

SEQ ID NO: 15 - DNA, 33 bp, Artificial
  APRIL D132A primer
tccaaggatgactccgctgtgacagaggtgatg

SEQ ID NO: 16 - DNA, 38 bp, Artificial
  APRIL D132F primer
acctccaaggatgactcctttgtgacagaggtgatgtg

SEQ ID NO: 17 - DNA, 38 bp, Artificial
  APRIL D132T primer
acctccaaggatgactccactgtgacagaggtgatgtg

SEQ ID NO: 18 - DNA, 39 bp, Artificial
  APRIL D173R primer
atagccaggtcctgtttcaacgcgtgactttcaccatgg

SEQ ID NO: 19 - DNA, 40 bp, Artificial
  APRIL V174R primer
ccaggtcctgtttcaagacaggactttcaccatgggtcag

SEQ ID NO: 20 - DNA, 40 bp, Artificial
  APRIL T175F primer
ggtcctgtttcaagacgtgtttttcaccatgggtcaggtg

SEQ ID NO: 21 - DNA, 41 bp, Artificial
  APRIL T175L primer
ggtcctgtttcaagacgtgctattcaccatgggtcaggtgg

SEQ ID NO: 22 - DNA, 36 bp, Artificial
  APRIL R233A primer
gtgtcataattccccgggcagcggcgaaacttaacc

SEQ ID NO: 23 - DNA, 40 bp, Artificial
  APRIL R233E primer
gagtgtcataattccccgggcagaggcgaaacttaacctc

SEQ ID NO: 24 - DNA, 38 bp, Artificial
  APRIL H241T primer
cgaaacttaacctctctccaactggaaccttcctgggg

SEQ ID NO: 25 - DNA, 35 bp, Artificial
  APRIL D132Y primer
cctccaaggatgactcctatgtgacagaggtgatg

SEQ ID NO: 26 - DNA, 40 bp, Artificial
  APRIL  T175Y primer
ggtcctgtttcaagacgtgtatttcaccatgggtcaggtg

SEQ ID NO: 27 - DNA, 40 bp, Artificial
  APRIL  T192R primer
ggccaaggaaggcaggagaggctattccgatgtataagaa

SEQ ID NO: 28 - DNA, 40 bp, Artificial
  APRIL  A232L primer
gagtgtcataattccccggttaagggcgaaacttaacctc

SEQ ID NO: 29 - DNA, 40 bp, Artificial
  APRIL  T175D primer
ggtcctgtttcaagacgtggatttcaccatgggtcaggtg

SEQ ID NO: 30 - DNA, 28 bp, Artificial
  APRIL  R206E primer
ctcccacccggacgaggcctacaacagc

SEQ ID NO: 31 - DNA, 29 bp, Artificial
  APRIL  D205K primer
gccctcccacccgaagcgggcctacaaca

SEQ ID NO: 32 - DNA, 29 bp, Artificial
  APRIL  D205Y primer
gccctcccacccgtatcgggcctacaaca

SEQ ID NO: 33 - DNA, 30 bp, Artificial
  APRIL  R206M primer
cctcccacccggacatggcctacaacagct

SEQ ID NO: 34 - protein, 38 aa, Artificial
  ECD ligand binding domain of human TACI
SLSCRKEQGKFYDHLLRDCISCASICGQHPKQCAYFCE

SEQ ID NO: 35 - protein, 36 aa, Artificial
  ECD ligand binding domain of human BCMA
CSQNEYFDSLLHACIPCQLRCSSNTPPLTCQRYCAN

SEQ ID NO: 36 - protein, 146 aa, Artificial
  extracellular ligand binding domain of human APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 37 - protein, 146 aa, Artificial
  extracellular ligand binding domain of murine APRIL
AVLTQKHKKKHSVLHLVPVNITSKADSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 38 - protein, 35 aa, Artificial
  human TACI APRIL ligand domain
CRKEQGKFYDHLLRDCISCASICGQHPKQCAYFCE

SEQ ID NO: 39 - protein, 35 aa, Artificial
  murine TACI APRIL ligand domain
CRKEQGRYYDHLLGACVSCDSTCTQHPQQCAHFCE

SEQ ID NO: 40 - protein, 36 aa, Artificial
  human BCMA APRIL ligand domain
CSQNEYFDSLLHACIPCQLRCSSNTPPLTCQRYCNA

SEQ ID NO: 41 - protein, 34 aa, Artificial
  murine BCMA APRIL ligand domain
CFHSEYFDSLLHACKPCHLRCSNPPATCQPYCDP

(19) United States
(12) Patent Application Publication
(10) Pub. No.: US 2014/0178329 A1
(43) Pub. Date: Jun. 26, 2014

(54) VARIANTS OF THE TNF SUPERFAMILY AND USES THEREOF

(71) Applicants: Institucio Catalana de Recerca I Estudis Avancats (ICREA), Barcelona (ES); Fundacio Privada Centre de Regulacio Genomica (CRG), Barcelona (ES)

(72) Inventors: Luis Serrano Pubul, Barcelona (ES); Albert Martinus Van Der Sloot, Montreal (CA); Jan Paul Medema, Amsterdam (NL); Fiona Clare Kimberley, Teddington (GB)

(73) Assignees: Institucio Catalana de Recerca I Estudis Avancats (ICREA), Barcelona (ES); Fundacio Privada Centre de Regulacio Genomica (CRG), Barcelona (ES)

(21) Appl. No.: 14/105,392

(22) Filed: Dec. 13, 2013

Related U.S. Application Data
(60) Provisional application No. 61/739,941, filed on Dec. 20, 2012.

(51) Int. Cl. C07K 14/525

(52) U.S. Cl. CPC C07K 14/525
USPC 424/85.1; 530/350; 536/23.5; 435/320.1; 435/366; 435/69.5; 435/375; 435/325

(57) ABSTRACT

Described are novel variants of APRIL that modulate signaling via receptor-specific agonist activity, and nucleic acids encoding these variant proteins. Further described is the use of these novel proteins in the treatment of APRIL-associated disorders, in particular, pathologies of the immune system and oncological disorders.

### CROSS-REFERENCE TO RELATED APPLICATION

This application claims the benefit under 35 U.S.C. §119(e) of U.S. Provisional Patent Application Ser. No. 61/739,941, filed Dec. 20, 2012, the disclosure of which is hereby incorporated herein in its entirety by this reference.

### TECHNICAL FIELD

The disclosure relates to biotechnology generally and more specifically to novel variants of APRIL, which modulate the signaling toward specific receptor agonist activity, and nucleic acids encoding these variant proteins. It further relates to the use of these novel proteins in the treatment of APRIL-associated disorders, in particular, pathologies of the immune system and oncological disorders.

### BACKGROUND

A proliferation inducing ligand (APRIL) is a member of the TNF ligand superfamily originally described as a tumor-promoting factor.⁽¹⁾ Physiologically, it plays a prominent role in humoral immunity, in particular, by driving antibody class-switch toward IgG and IgA,^{(2, 3)} and by promoting survival of plasma cells.⁽⁴⁾ APRIL has also been identified as a pro-survival factor for several B cell malignancies, possibly via the activation of transcription factor NF-κB (reviewed in reference (5)). APRIL is also thought to promote tumor formation in a number of solid malignancies, either indirectly via infiltrating cells or directly via autocrine stimulation of the tumor itself.^{(1, 6, 7)} In line with these results, we recently identified a clear role for APRIL in supporting tumorigenesis in the gastrointestinal tract.⁽⁸⁾

As most TNF ligand factors, APRIL is synthesized as a precursor protein, which is further processed into a mature form. Biologically active APRIL is secreted following intracellular processing in the Golgi apparatus by furin convertase, an enzyme that cleaves the full length APRIL protein (250 amino acids) between amino acids 104-105 (numbering according to UniprotKB/SwissProt entry 075888) and releases the soluble, extracellular receptor-binding region (amino acids 105-250) from the propeptide region (amino acids 1-104).⁽³⁹⁾ A membrane-bound form of APRIL is generated by an alternative splicing event in which a fusion is generated between the extracellular portion of APRIL (amino acids 86-250) and the transmembrane and intracellular portion of TNF-related weak inducer of apoptosis (TWEAK) (amino acids 1-165)—the end product named TWE-PRIL. However, the biological relevance and expression of TWE-PRIL is little understood.^{(40, 41, 42)}

APRIL binds two different receptors of the TNF receptor superfamily: B cell maturation antigen (BCMA), and transmembrane activator and cyclophilin ligand interactor (TACI), which are also bound by its homolog B cell-activating factor (BAFF).⁽⁹⁻¹²⁾ In addition, APRIL binds to heparan sulphate proteoglycans (HSPGs), which appear to play a predominantly structural role by enabling APRIL cross-linking,^{(13, 14)} although a distinct signaling role in different contexts cannot be eliminated. In addition to APRIL, TACI can also bind to HSPGs, which is suggested to lead to its activation.⁽¹⁵⁾ All these potential binding partners make it difficult to unravel APRIL signaling in a given context, and to assess the individual contributions of TACI and BCMA. Therefore, it is not surprising that little is known about the individual signaling pathways activated in response to signals via each of the APRIL receptors, or precisely how these are separated in terms of the formation of distinct intracellular complexes and recruitment of signaling adaptors. Much of what is currently known with regard to activation of transcription factors and recruitment of internal adaptors, such as TNF-receptor associated factors (TRAFs), has been carried out using transfection studies^{(16, 17)} or RNAi-mediated knock-down studies,⁽¹⁸⁾ which pose possible problems associated with over-expression or simultaneous removal of multiple interactions, respectively. A need exists in the art to develop APRIL variants that can interfere with distinct intracellular signaling processes.

### DISCLOSURE

Thus, it was an aim hereof to develop such APRIL variants with receptor-interaction domains that are modified, such that each domain has either i) significantly reduced affinity and concomitant reduction in signaling capacity for one or more cognate receptors, ii) significantly enhanced affinity and concomitant increase in signaling capacity for one of its cognate receptors, or iii) a combination of i) and ii). More specifically, variants of the soluble, extracellular region of APRIL including the receptor-interaction domain modified as described herein have been produced and used.

In the disclosure, generated were variant forms of the APRIL protein and tested their ability to bind either BCMA or TACI. Six mutants (or variants, which is here used as an equivalent term) were of particular interest: APRIL-R206E, APRIL-R206M, APRIL-T175D, and APRIL-D205Y, which showed clear specificity toward both human and mouse BCMA, and APRIL-D132F and APRIL-D132Y, which showed considerable selectivity for TACI. Following initial ELISAs using immobilized receptors, we further confirmed the binding characteristics in the context of cell-based assays, using either transfected cells in which receptors were over-expressed, or endogenously expressing BCMA or TACI cell species. Finally, we used these APRIL variants in a B cell assay to show distinct roles for TACI and BCMA in B cell function.

Accordingly, provided are variants of the extracellular domain of APRIL ligand proteins that modulate the bioactivity of APRIL receptors. Provided are variant APRIL proteins that comprise an amino acid sequence (“peptide”) that has one modification as compared to the naturally occurring APRIL protein sequence.

In a specific aspect, provided is an APRIL variant polypeptide comprising a polypeptide sequence selected from the list consisting of SEQ ID NOS:2, 4, 6, 8, 10 or 12, or a fragment corresponding to amino acids 104-250 or 105-250 in SEQ ID NOS:2, 4, 6, 8, 10 or 12.

In yet another aspect, provided is a recombinant nucleic acid encoding a variant APRIL polypeptide hereof.

In a particular embodiment, a recombinant nucleic acid encoding a fragment of SEQ ID NOS:2, 4, 6, 8, 10 or 12 corresponds with a nucleic acid sequence starting at nucleotide 310 or 313 and ending at nucleotide 750 in SEQ ID NOS:1, 3, 5, 7, 9 or 11.

In the disclosure, the terms “polypeptide” and “protein” are equivalent terms with the same meaning.

In yet another aspect, provided is an expression vector comprising a recombinant nucleic acid hereof.

In yet another aspect, provided is a host cell comprising a recombinant nucleic acid hereof.

In yet another aspect, provided is a host cell comprising an expression vector hereof.

In yet another aspect, provided is a method for producing a variant APRIL polypeptide hereof comprising culturing a recombinant host cell under conditions suitable for expression of a nucleic acid hereof.

In yet another aspect, an APRIL variant polypeptide hereof is used for the treatment of an APRIL-associated disorder.

In yet another aspect, provided is a pharmaceutical composition comprising an APRIL variant polypeptide hereof and a pharmaceutically acceptable carrier.

In yet another aspect, provided is a pharmaceutical composition for the treatment of an APRIL-associated disorder.

In yet another aspect, provided are cell lines and animal models comprising APRIL variant nucleotide sequences hereof.

In yet another embodiment, provided is the use of the APRIL polypeptide variants hereof for in vivo or in vitro or ex vivo treatment applications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Crystal Structures of APRIL in complex with BCMA and TACI and prediction of the APRIL selective mutants. (Panel A) Front view of APRIL (light and dark green) in complex with TACI (orange) or BCMA (blue). The APRIL-TACI and APRIL-BCMA complexes are superimposed. APRIL monomers are depicted using a molecular surface representation and main chain coordinates of the receptors are depicted in cartoon style. For clarity only, a single receptor unit is depicted and two ligand monomers are shown. The TACI and BCMA receptor-binding interface of APRIL is mapped in red on the APRIL surface. In contrast to most other TNF family ligands, the receptor-binding interface resides only for a small part in the cleft between two adjacent ligand monomers, as most of the interaction surface is located on the central surface of a single APRIL monomer. (Panel B) Detailed view of TACI and BCMA in complex with APRIL. Selected APRIL residues involved in interaction with the receptors are depicted (c-atoms of APRIL in complex with BCMA or TACI are light green or dark green, respectively). (Panel C) Detailed view of TACI (orange) and BCMA (blue) residues involved in APRIL binding. TACI and BCMA show a RMSD of 1.45 Å upon superposition (calculated over 95 main chain atoms), the main chain coordinates show a larger displacement C-terminally of the beta-sheet. The interacting residues of BCMA or TACI are relatively non-conserved. Labels of BCMA residues are colored blue and TACI in black, cysteine bridges are colored yellow. (Panel D) Structure-based alignment of the ECD ligand-binding domain of human TACI (SEQ ID NO:34) and human BCMA (SEQ ID NO:35). Brackets indicate cysteine bridge connectivity. Full bars depict conserved residues. (Panel E) FoldX interaction energy. Interaction-free energy between APRIL variants and BCMA or TACI is calculated as the difference with the interaction energy of wild-type APRIL and expressed as ΔΔG in kcal/mol. The FoldX interaction energy is corrected for unfavorable intra-chain Van der Waals clashes upon mutation (see methods). Variants are grouped as TACI-specific or BCMA-specific. R231A, a previously constructed APRIL variant unable to bind both receptors, was used as control. Structure images were generated using Pymol (on the WorldWideWeb at pymol.org) and based on PDB IDs 1xu1 and 1xu2.⁽²¹⁾

FIG. 2: Production and real binding properties of the APRIL mutants. (Panel A) Production of APRIL variants in conditioned medium by transient transfection of 293T cells. Supernatants (10 μl of each) were analyzed by anti-FLAG immunoblotting. Upper panel: mutants predicted to be selective for BCMA. Lower panel: mutants predicted to be selective for TACI. All mutants were checked more than three times following independent rounds of transfection to assess their expression. (Panels B and C) Receptor binding ELISA to compare binding of the predicted BCMA- and TACI-specific APRIL variants to human BCMA-Fc and TACI-Fc. Bars, from left to right, represent doubling dilutions of the conditioned media starting from undiluted media. Relevant APRIL variants are shown with dark grey bars, WT APRIL with black bars and other variants with light grey bars. This is representative of three separate experiments performed with independent APRIL-containing cell-conditioned media. R231A APRIL variant does not bind any of the APRIL receptors (negative binding control).

FIG. 3: R206E shows specificity for BCMA while D132Y and D132F show selectivity for TACI. Binding activity of the ligands was tested on TACI:Fas and BCMA:Fas expressing reporter cells. The ligand binding is directly associated with induced cell death. (Panel A) Staining for human BCMA and TACI on Jurkat BCMA:Fas (left) and Jurkat JOM2 TACI:Fas cells (right). (Panels B and C) Measurement of cell death produced after 16 hours treatment with doubling dilutions of the APRIL variants on BCMA:Fas (Panel B) and TACI:Fas (Panel C) reporter cells. (Panel D) Microscopic pictures (40×) of Jurkat-BCMA-Fas (top) and Jurkat-TACI-Fas (bottom) cells after one hour stimulation with the indicated APRIL variants. Conditioned media were matched for APRIL amounts before incubation.

FIG. 4: D132F and D132Y but not R206E, triggered TACI internalization on endogenously expressed receptors. Cells were stained with a PE-coupled anti-TACI antibody and incubated with the indicated ligands for 1 hour at 37° C. to allow receptor internalization. Subsequently, cells were placed on ice to halt membrane movements and then treated with either PBS (control) or acid solution (pH 2) to strip off labeled receptors that were not internalized. (Panel A) Example of FACS profile of the TACI internalization for A20 cells. The high PE signal that remained after acid treatment (marked boxes) reflects TACI being internalized and protected inside cells. (Panel B) Quantification of FIG. 4, Panel A, expressed as % of APRIL induced TACI internalization. (Panel C) Quantification of TACI internalization for human Raji cells.

FIG. 5: Differential effects of APRIL variants on B splenocytes survival and IgA production. Primary mouse splenocytes were positively selected for B220 and stimulated for six days with the indicated ligands in conditioned medium diluted 1:1 in normal medium. After six days, PI negative cells (live cells) were counted (Panel A) and supernatants were screened for soluble IgA levels (Panel B). (Panel C) Graphs representing the ratio between IgA and number of live B cells stimulated. Due to the different concentrations of ligands produced in conditioned medium, the concentration of all the variants were adjusted to that of the lowest expresser, D132F. Error bars represent SEM among triplicates.

FIG. 6: (Panel A) Structural consequences of the R206E substitution. In the WT APRIL-TACI crystal structure, R206 makes hydrogen bonds with TACI, whereas in the WT APRIL-BCMA crystal structure, R206 is not involved in the interaction with BCMA. The E206 substitution in TACI and BCMA is not involved in hydrogen bond interactions. (Panel B) Structural consequences of the D132F and D132Y substitution. In the WT APRIL-BCMA structure, Asp132 (D132) is involved in a favorable electrostatic interaction with Arg27, whereas in the WT APRIL-TACI complex, Asp132 accepts a (weak) hydrogen bond from Gln99 (Q99) of TACI. The loss of this hydrogen bond due to the F132 and Y132 substitution is compensated in TACI by favorable Van der Waals' interactions, whereas in BCMA, the Phe or Tyr either clashes with Arg27 (R27) or with the main chain oxygen of Ser131 (S131) of APRIL. TACI is depicted in orange, BCMA in blue and APRIL in green. The D132F and D132Y structures are superimposed; residues that differ are indicated in lighter shades of blue, green and orange. Hydrogen bonds are shown as an orange dotted line and Van der Waals clashes are shown as a black dotted line.

FIG. 7: Comparison of human and murine APRIL, BCMA and TACI sequence identity. (Panel A) Sequence alignment of the extracellular ligand-binding domain of human APRIL (SEQ ID NO:36) and murine APRIL (SEQ ID NO:37). Percentage sequence identity between the extracellular ligand-binding domain of human APRIL (residues 105-250) (SEQ ID NO:36) and murine APRIL (residues 96-241) (SEQ ID NO:37) is 86%. (Panel B) Top: Sequence alignment of the human TACI (residues 68-115) (SEQ ID NO:38) and murine TACI (residues 43-77) (SEQ ID NO:39) APRIL ligand domain. Bottom: Sequence alignment of the human BCMA (residues 8-43) (SEQ ID NO:40) and murine BCMA (residues 5-38) (SEQ ID NO:41) APRIL ligand domain. The percentage sequence identity is 71% and 67%, respectively. Full height bars indicate conserved positions and half height bars indicate non-conserved positions.

FIG. 8: Western blot of soluble protein and cell lysates. Anti-FLAG western blot analysis of supernatants and cell lysates of 293T cells transfected with the indicated APRIL variants.

FIG. 9: Receptor-binding ELISA using murine TACI-Fc and BCMA-Fc coated plates. Receptor-binding ELISA comparing the binding of the predicted BCMA and TACI-specific APRIL variants to both murine BCMA-Fc and TACI-Fc.

FIG. 10: Surface Plasmon Resonance sensorgrams to illustrate binding of TACI-Fc and BCMA-Fc to WT and R206E-APRIL. Sensorgrams of human (Panel A) and mouse (Panel B) receptors binding to WT and R206E-APRIL. Stepwise increments in the curves represent sequential injections of receptor at the following concentrations: 1, 5, 10 and 25 nM. In some cases, no value for affinity was calculated due to a low binding response or high drift.

FIG. 11: TACI internalization using confocal microscopy. Microscopic pictures representing formation of TACI clusters on A20 cells after one hour incubation with either APRIL-WT (WT) or control medium (MOCK).

FIG. 12: B cell stimulation of APRIL WT and R206E at a higher concentration. Primary mouse splenocytes were positively selected for B220 and stimulated for six days with the indicated ligands in conditioned medium diluted 1:1 in normal medium. The concentration of APRIL WT, R231A and R206E were matched to each other and were around 30 times more concentrated than the concentration used in FIG. 5. (Panel A) Number of PI negative cells (live cells); (Panel B) Quantification of soluble IgA. Error bars represent SEM among triplicates.

### DETAILED DESCRIPTION

The disclosure will be described with respect to particular embodiments and with reference to certain drawings but the disclosure is not limited thereto, but only by the claims. Any reference signs in the claims shall not be construed as limiting the scope. The drawings described are only schematic and are non-limiting. Where the term “comprising” is used in the present description and claims, it does not exclude other elements or steps. Where an indefinite or definite article is used when referring to a singular noun, e.g., “a,” “an,” or “the,” this includes a plural of that noun unless something else is specifically stated. Furthermore, the terms “first,” “second,” “third,” and the like, in the description and in the claims, are used for distinguishing between similar elements and not necessarily for describing a sequential or chronological order. It is to be understood that the terms so used are interchangeable under appropriate circumstances and that the embodiments hereof described herein are capable of operation in other sequences than described or illustrated herein.

The following terms or definitions are provided solely to aid in the understanding hereof. Unless specifically defined herein, all terms used herein have the same meaning as they would to one skilled in the art. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 100), John Wiley & Sons, New York (2012), for definitions and terms of the art. The definitions provided herein should not be construed to have a scope less than understood by a person of ordinary skill in the art.

The proliferation-inducing ligand (APRIL) (also known as TRDL-1 alpha, TALL-2, TNFSF13) and its closest homologue, BAFF (also known as B-cell Activation Factor, BLyS, TALL-1, THANK, zTNF4 and TNFSF13B), are members of the TNF super family (TNFSF) of proteins. The prototype of the family, Tumor Necrosis Factor Alpha (TNFA), originally discovered for its in vivo effect causing tumors to regress, is a key mediator of inflammation. BAFF and APRIL proteins participate in a variety of cellular and intracellular signaling processes, and are synthesized as type 2 membrane proteins that fold into conserved beta-sheet structures and can be cleaved intracellularly or from the membrane to be secreted as soluble forms. APRIL is also implicated in several cancers as a pro-survival factor. APRIL binds two different TNF receptors: B cell maturation antigen (BCMA), and transmembrane activator and cyclophilin ligand interactor (TACI), and also interacts independently with heparan sulfate proteoglycans (HSPGs). As APRIL shares binding of the TNF receptors with B cell activation factor (BAFF), separating the precise signaling pathways activated by either ligand in a given context has proven quite difficult. The human APRIL DNA Genbank sequence record is AF046888. Residue numbering of the variants is based on the human APRIL protein sequence (also depicted in SEQ ID NO:14) as described in the Expasy Uniprot record (O75888).

SEQ ID NO:1 depicts the nucleotide sequence of the full-length APRIL-R206E variant.

SEQ ID NO:2 depicts the amino acid sequence of the full-length APRIL-R206E variant.

SEQ ID NO:3 depicts the nucleotide sequence of the full-length APRIL-D132F variant.

SEQ ID NO:4 depicts the amino acid sequence of the full-length APRIL-D132F variant.

SEQ ID NO:5 depicts the nucleotide sequence of the full-length APRIL-D132Y variant.

SEQ ID NO:6 depicts the amino acid sequence of the full-length APRIL-D132Y variant.

SEQ ID NO:7 depicts the nucleotide sequence of the full-length APRIL-R206M variant.

SEQ ID NO:8 depicts the amino acid sequence of the full-length APRIL-R206M variant.

SEQ ID NO:9 depicts the nucleotide sequence of the full-length APRIL-D205Y variant.

SEQ ID NO:10 depicts the amino acid sequence of the full-length APRIL-D205Y variant.

SEQ ID NO:11 depicts the nucleotide sequence of the full-length APRIL-T175D variant.

SEQ ID NO:12 depicts the amino acid sequence of the full-length APRIL-T175D variant.

SEQ ID NO:13 depicts the nucleotide sequence of the full-length wild-type APRIL

SEQ ID NO:14 depicts the amino acid sequence of the full-length wild-type APRIL.

Four BCMA-specific variants of APRIL were successfully generated: APRIL-R206E, APRIL-R206M, APRIL-T175D and APRIL-D205Y and two TACI-selective variants: D132F and D132Y. These six different APRIL variants show selective activity toward their receptors in several in vitro assays. Moreover, in the disclosure, we show through these APRIL variants that BCMA and TACI have a distinct role in APRIL-induced B cell stimulation.

In certain embodiments, a mutant APRIL nucleic acid encodes a mutant APRIL protein. As will be appreciated by those in the art, due to the degeneracy of the genetic code, an extremely large number of nucleic acids may be made, all of which encode a specific variant APRIL protein of the disclosure. Thus, having the six particular amino acid sequences of the variants hereof, those skilled in the art could make any number of different nucleic acids, by simply modifying the sequence of one or more codons in a way that does not change the amino acid sequence of the variant APRIL proteins.

In certain embodiments, the variant APRIL proteins and nucleic acids are recombinant. As used herein, “nucleic acid” may refer to either DNA or RNA, or molecules that contain both desoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments. The nucleic acid may be double-stranded, single-stranded, or contain portions of both double-stranded or single-stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases or through, for example, gene synthesis, in a form not normally found in nature. Thus, an isolated variant APRIL nucleic acid, in a linear form, or an expression vector formed in vitro by ligating DNA molecules that are not normally joined, are both considered recombinant for the purposes hereof. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purposes hereof. A “recombinant protein” is a protein made using recombinant techniques, i.e., through the expression of a recombinant nucleic acid. A recombinant protein is distinguished from naturally occurring protein by at least one or more characteristics. For example, the protein may be isolated or purified away from some or all of the proteins and compounds with which it is normally associated in its wild-type host, and thus may be substantially pure. A substantially pure protein comprises at least about 75% by weight of the total protein, with at least about 80% being preferred, and at least about 90% being particularly preferred. The definition includes the production of a variant APRIL protein from one organism in a different organism or host cell. Alternatively, the protein may be made at a significantly higher concentration than is normally seen, through the use of an inducible promoter or high expression promoter, such that the protein is made at increased concentration levels.

In certain embodiments, variant APRIL proteins may be prepared by in vitro synthesis using established techniques (e.g., chemical synthesis; see, for example, Wilken et al., Curr. Opin. Biotechnol. 9:412-26 (1998)).

Using the nucleic acids hereof, which encode a variant APRIL protein, a variety of expression vectors can be prepared. The expression vectors may be either self-replicating extrachromosomal vectors or vectors that integrate into a host genome. Generally, these expression vectors include transcriptional and translational regulatory nucleic acids operably linked to the nucleic acid encoding the variant APRIL protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. In general, the transcriptional and translational regulatory sequences may include, but are not limited to, promoter sequences, ribosomal binding sites, transcriptional start and stop sequences, translational start and stop sequences, and enhancer or activator sequences. In certain embodiments, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the disclosure. In certain embodiments, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example, in mammalian or insect cells for expression and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs and methods for integrating vectors are well known in the art.

In addition, in certain embodiments, the expression vector contains a selectable marker gene to allow the selection of transformed host cells. Selection genes are well known in the art and will vary with the host cell used.

In certain embodiments, the expression vector comprises the components described above and a gene encoding a variant APRIL protein. As will be appreciated by those in the art, all combinations are possible and accordingly, as used herein, the combination of components, comprised by one or more vectors, which may be retroviral or not, is referred to herein as a “vector composition.”

In particular embodiments, the variant APRIL proteins of the disclosure are produced by culturing a host cell transformed with an expression vector containing nucleic acid encoding a variant APRIL protein, under the appropriate conditions to induce or cause expression of the variant APRIL protein. The conditions appropriate for variant APRIL protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. Appropriate host cells include yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, 293 cells, CHO, COS, Pichia pastoris and the like.

In certain embodiments, the variant APRIL proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the protein into mRNA. A promoter will have a transcription-initiating region, which is usually placed proximal to the 5′ end of the coding sequence, and a TATA box located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

In another embodiment, provided is a cell line (such as a mammalian cell line) comprising a nucleotide sequence encoding a variant APRIL polypeptide hereof. In another particular embodiment, provided is a transgenic non-human animal (e.g., a mouse or a rat) comprising a nucleotide sequence encoding a variant APRIL polypeptide hereof. In a particular embodiment (part of), one or both of the WT alleles in human or other mammalian (e.g., stem) cells can be replaced with a variant encoding sequence using genome/gene-editing technology (zinc finger nucleases, mega-nucleases, TALEN, etc.).

In another particular embodiment, the variant APRIL proteins are used for the manufacture of a medicament to treat APRIL-associated diseases.

In certain embodiments, the variant APRIL proteins are used to treat APRIL-associated diseases.

Variant APRIL proteins hereof can be expressed as chimeric proteins, fused to other functional protein domains in order to further enhance it therapeutic efficacy. For example, a fusion with a trimerizing domain can enhance the stability, reduce clearance and improve pharmacokinetics and half-life of APRIL variant proteins. Similarly, fusion with a serum albumin binding domain can reduce clearance and improve pharmacokinetic parameters such as half-life. Fusion with a toxin can be used to selectively target and destroy TACI or BCMA-expressing cells. Variant APRIL proteins might also be expressed as fusions with a domain that selectively targets a specific subset of cells, for example, cancer cells.

Variant APRIL proteins or variant APRIL chimeric proteins can also be modified by pegylation or particular forms of glycosylation in order to improve half-life and stability or reduce immunogenicity. As will be appreciated, modifications such as pegylation will be achieved by chemical means, whereas glycosylation can be achieved chemically or biologically.

The term “APRIL-associated diseases” comprises congestive heart failure (CHF), skin diseases (e.g., acne, eczema), myocarditis and other conditions of the myocardium, systemic lupus erythematosus, diabetes, spondylopathies, multiple myeloma, breast cancer, lung cancer, kidney cancer and rectal cancer; bone metastasis, ankylosing spondylitis, transplant rejection, hematologic neoplasias and neoplastic-like conditions, for example, Hodgkin's lymphoma; non-Hodgkin's lymphomas (Burkitt's lymphoma, small lymphocytic lymphoma/chronic lymphocytic leukemia, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemangiopericytoma), rheumatoid arthritis, inflammatory bowel diseases (IBD), sepsis and septic shock, Crohn's Disease, psoriasis, schleraderma, graft versus host disease (GVHD), allogenic islet graft rejection, hematologic malignancies, such as multiple myeloma (MM), myelodysplastic syndrome (MDS) and acute myelogenous leukemia (AML), cancer and the inflammation associated with tumors, peripheral nerve injury or demyelinating diseases.

### Use of APRIL-Variants as a Polypeptide for the Manufacture of a Medicament for Treatment of APRIL-Associated Diseases

The term “medicament to treat” relates to a composition comprising APRIL-variants as described above and a pharmaceutically acceptable carrier or excipient (both terms can be used interchangeably) to treat APRIL-associated diseases. Suitable carriers or excipients known to the skilled person are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers (Gennaro (2000) Remington: The Science and Practice of Pharmacy 20th ed, ISBN: 0683306472). The “medicament” may be administered by any suitable method within the knowledge of the skilled person. The preferred route of administration is parenterally. In parental administration, the medicament hereof will be formulated in a unit dosage injectable form such as a solution, suspension or emulsion, in association with the pharmaceutically acceptable excipients as defined above. However, the dosage and mode of administration will depend on the individual and the particular indication. Generally, the medicament is administered so that the protein, polypeptide, or peptide of the disclosure is given at a dose between 1 μg/kg and 10 mg/kg, more preferably between 1 μg/kg and 5 mg/kg, most preferably between 1 and 100 μg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute. It is clear to the person skilled in the art that the use of a therapeutic composition comprising an APRIL-variant for the manufacture of a medicament to treat APRIL-associated diseases can be administered by any suitable means, including but not limited to, parenteral, subcutaneous, intraperitoneal, intrapulmonary, and intranasal administration. Parenteral infusions include intramuscular, intravenous, intra-arterial, intraperitoneal, or subcutaneous administration. In addition, the therapeutic composition is suitably administered by pulse infusion, particularly with declining doses of the APRIL-variant protein. Preferably, the therapeutic composition is given by injections, most preferably intravenous or subcutaneous injections, depending in part on whether the administration is brief or chronic.

In a particular embodiment, the APRIL variants hereof can be used for in vivo treatment. The latter is discussed extensively hereinbefore.

In yet another particular embodiment, the APRIL variants hereof can be used for ex vivo treatment. The term “ex vivo treatment” implies that cells (e.g., orthologous cells or cells derived from a patient) are treated with an APRIL variant hereof for a certain amount of time and these treated cells are then subsequently brought back into a patient (e.g., through injection or other means of delivery). Applications for ex vivo therapy envisage the use of APRIL variants for expanding, selecting or differentiating blood cells (e.g., stem cells) that can be used for autologous transplantations or transfusions.

### Use of an APRIL-Variant as a Nucleic Acid

In a specific embodiment, nucleic acids comprising sequences encoding APRIL-variants are administered to treat APRIL-associated diseases by way of gene therapy. Gene therapy refers to therapy performed by the administration to a subject of an expressed or expressible nucleic acid. In this embodiment hereof, the nucleic acids produce APRIL-variants to treat APRIL-associated diseases. Any of the methods for gene therapy available in the art can be used according to the disclosure. Exemplary methods are described below. In case a nucleic acid sequence or a portion thereof capable of encoding an APRIL-variant is used for the manufacture of a medicament to treat APRIL-associated diseases, the medicament is preferably intended for delivery of the nucleic acid sequence into the cell, in a gene therapy treatment. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. A preferred method of non-viral delivery is via direct electroporation in muscle cells. Lipofection is described in, e.g., U.S. Pat. Nos. 5,049,386, 4,946,787, and 4,897,355, and lipofection reagents are sold commercially (e.g., TRANSFECTAM™ and LIPOFECTIN™). Cationic and neutral lipids that are suitable for efficient receptor-recognition lipofection of polynucleotides include those of Flegner, WO 91/17424, WO 91/16024. Delivery can be to cells (ex vivo administration) or directly to target tissues such as muscle fibers (in vivo administration). The preparation of lipid:nucleic acid complexes, including targeted liposomes such as immunolipid complexes, is well known to one of skill in the art (see, e.g., Crystal, 1995; Blaese et al., 1995; Behr, 1994; Remy et al., 1994; Gao and Huang, 1995; U.S. Pat. Nos. 4,186,183, 4,217,344, 4,235,871, 4,261,975, 4,485,054, 4,501,728, 4,774,085, 4,837,028, and 4,946,787). The use of RNA or DNA viral-based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral-based systems for the delivery of nucleic acids include, amongst others, retroviral, lentiviral, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues. In cases where transient expression of the nucleic acid is preferred, adenoviral-based systems, including replication-deficient adenoviral vectors, may be used. Adenoviral-based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors, including recombinant adeno-associated virus vectors, are also used to transduce cells with target nucleic acids, e.g., in the in vitro production of nucleic acids and peptides, and for in vivo and ex vivo gene therapy procedures (see, e.g., U.S. Pat. No. 4,797,368; WO 93/24641; Kotin, 1994; The construction of recombinant AAV vectors is described in a number of publications, including U.S. Pat. No. 5,173,414; Hermonat & Muzyczka, 1984; Samulski et al., 1989). Gene therapy vectors can be delivered in vivo by administration to an individual patient, typically by systemic administration (e.g., intravenous, intraperitoneal, intratracheal, subdermal, or intracranial infusion) or topical application. In a particular embodiment, intramuscular administration is preferred. In another embodiment, envisaged is the use of a hydrodynamic gene therapeutic method. Hydrodynamic gene therapy is disclosed in U.S. Pat. No. 6,627,616 (Minis Corporation, Madison) and involves the intravascular delivery of non-viral nucleic acids encoding APRIL-variants, whereby the permeability of vessels is increased through, for example, the application of an increased pressure inside the vessel or through the co-administration of vessel permeability increasing compounds such as, for example, papaverine.

Alternatively, vectors can be delivered to cells ex vivo, such as cells explanted from an individual patient (e.g., lymphocytes, bone marrow aspirates, tissue biopsy, myoblasts) or universal donor hematopoietic stem cells, followed by re-implantation of the cells into a patient, usually after selection for cells that have incorporated the vector. Ex vivo cell transfection for diagnostics, research, or for gene therapy (e.g., via re-infusion of the transfected cells into the host organism) is well known to those of skill in the art. In certain embodiments, cells are isolated from the subject organism, transfected with a nucleic acid (gene or cDNA), and re-infused back into the subject organism (e.g., patient). Various cell types suitable for ex vivo transfection are well known to those of skill in the art. In ex vivo transfection, the nucleic acid is introduced into a cell prior to administration in vivo of the resulting recombinant cell. Such introduction can be carried out by any method known in the art, including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the disclosure, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art. Cells into which a nucleic acid can be introduced for purposes of gene therapy encompass any desired, available cell type, and include, but are not limited to, epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular, hematopoietic stem or progenitor cells, e.g., as obtained from bone marrow, umbilical cord blood, peripheral blood, fetal liver, etc. In certain embodiments, the cell used for gene therapy is autologous to the patient. In an embodiment in which recombinant cells are used in gene therapy, nucleic acid sequences encoding APRIL-variants are introduced into the cells such that they are expressible by the cells or their progeny, and the recombinant cells are then administered in vivo for a therapeutic effect. In a specific embodiment, stem or progenitor cells (e.g., myoblasts) are used. In a specific embodiment, the nucleic acid to be introduced for purposes of gene therapy comprises an inducible promoter operably linked to the APRIL-variant coding region, such that expression of the nucleic acid is controllable by controlling the presence or absence of the appropriate inducer of transcription.

It is to be understood that although particular embodiments, specific configurations as well as materials and/or molecules, have been discussed herein for cells and methods according to the disclosure, various changes or modifications in form and detail may be made without departing from the scope and spirit hereof. The following examples are provided to better illustrate particular embodiments, and they should not be considered limiting the application.

### Examples

### 1. Computational Design of Receptor-Selective APRIL Variants

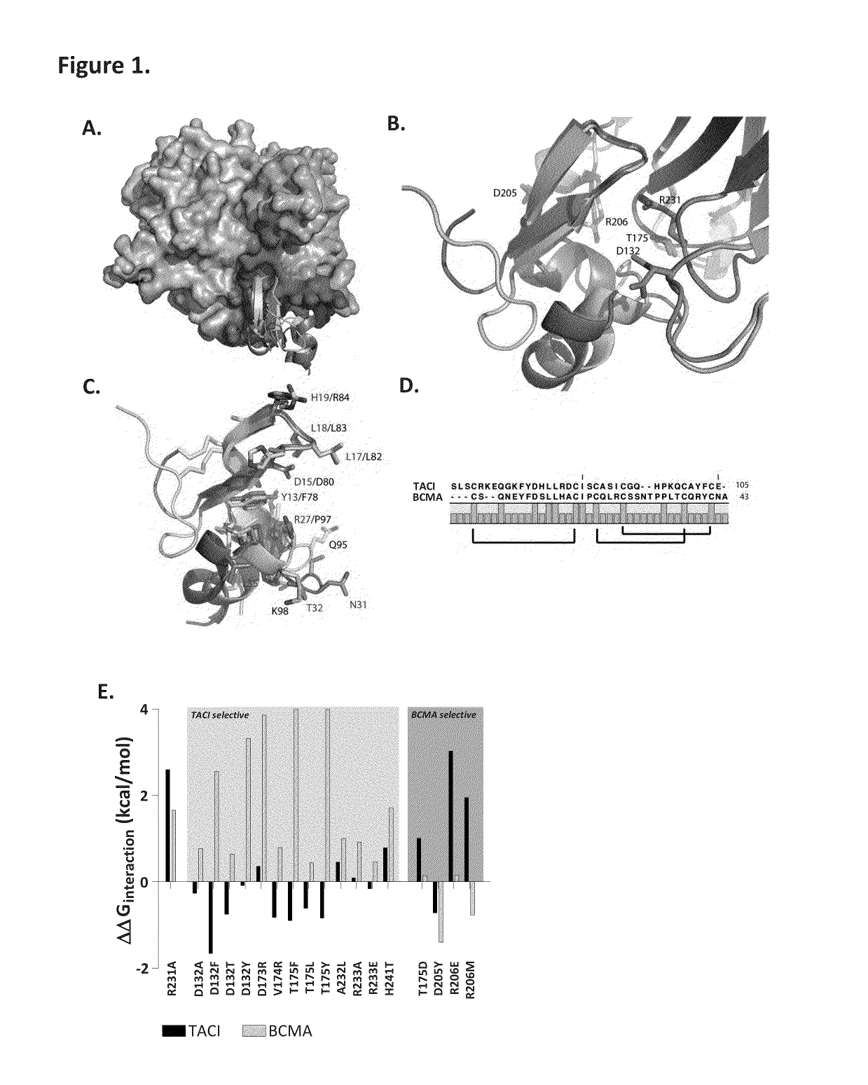

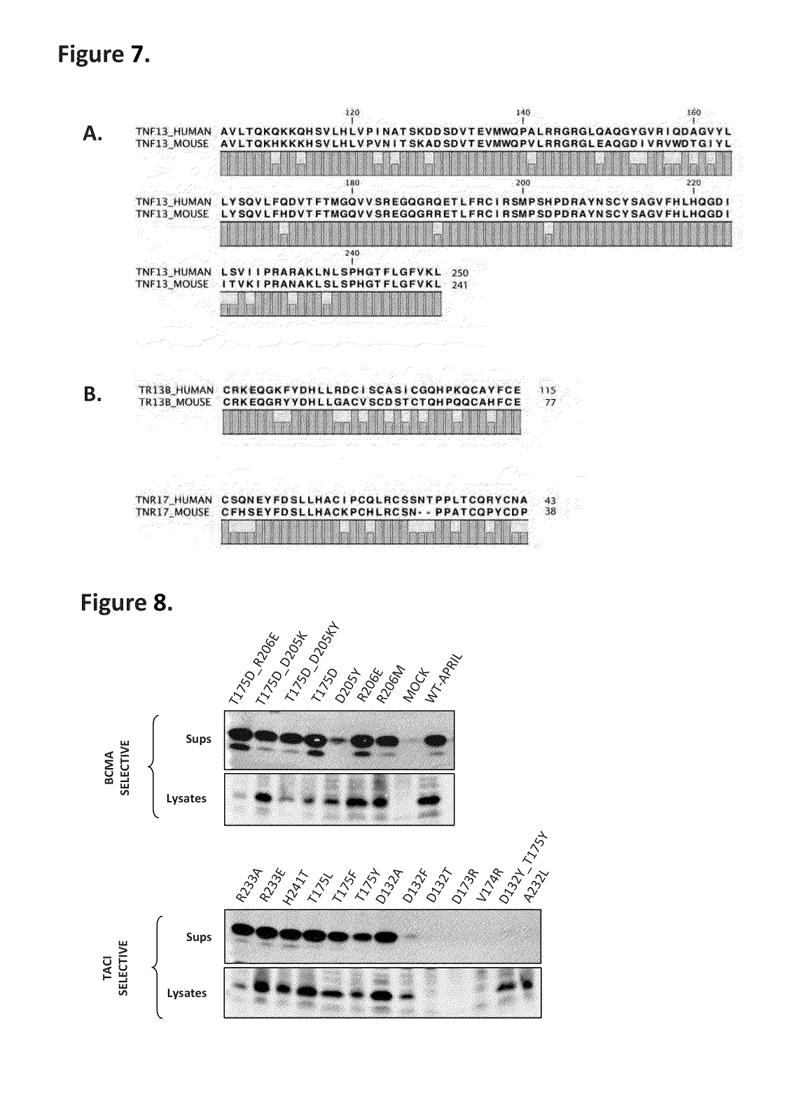

The X-ray crystal structures of murine APRIL, in complex with human TACI or human BCMA,⁽²¹⁾ were used as templates for designing receptor-selective variants of human APRIL. The ECD of murine APRIL shares 85% sequence identity with the human form. In addition, neither sequence contains an insertion or deletion relative to the other (FIG. 7, Panel A). Models of human APRIL in complex with TACI or BCMA were constructed by assuming an identical protein backbone conformation and by in silico mutating all non-conserved murine residues to its homologous human counterpart. A similar approach was successfully used in previous design work.^{(22, 23, 26)} Like most other TNF family ligands, APRIL is expressed as a homotrimer that binds three receptor monomers. In contrast to many other TNF ligands, the main interaction surface of a single receptor binding interface is not located in the cleft between two adjacent APRIL monomers, but instead resides mainly on the central surface of an APRIL monomer (FIG. 1, Panel A). The same can be observed for BAFF in complex with BAFF-R.⁽³³⁾ Inspection of the interface between APRIL and BCMA or TACI reveals that the main chain conformation of APRIL hardly changes upon interaction with the two different receptors, and that many side chains only show minor conformational changes. In contrast, the main chain conformation of TACI and BCMA show considerable deviation in the binding interface (FIG. 1, Panel B) and relatively few conserved interactions at the amino acid level (FIG. 1, Panels C and D), which is a favorable starting condition for the computational protein design approach. Due to the three-fold symmetry of the APRIL-receptor complex, a “design unit” consisting of only two adjacent APRIL monomers and a single receptor monomer was used in the design process. Residues comprising the receptor interface of APRIL were identified and each of these residues was subsequently mutated into all other 19 naturally occurring amino acids, and their contribution to the interaction energy was calculated by the FoldX protein design algorithm. Evaluation of the calculated interaction energy revealed several mutations that could confer APRIL receptor selectivity toward BCMA or TACI. Subsequently, several combinations of single BCMA or TACI specificity conferring mutants were combined in single APRIL variants to evaluate the effect on receptor binding by FoldX. The best performing single mutants and combination mutants were selected for experimental characterization (FIG. 1, Panel E). For the purpose of clarity, APRIL mutants will be referred to by only the amino acid substitution (e.g., R231A means APRIL-R231A).

### 2. Generation of APRIL Variants

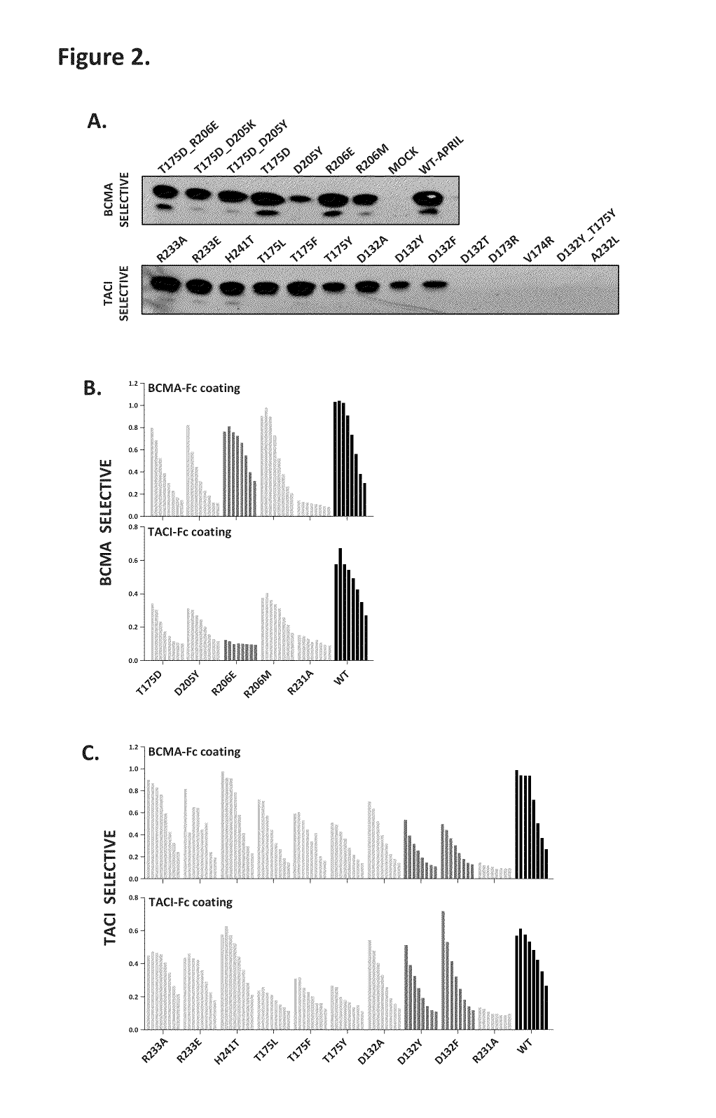

As human WT-APRIL does not express efficiently as a soluble recombinant protein in Escherichia coli and is difficult to purify with high yield from mammalian cell cultures, it was decided to test FLAG-tagged APRIL variants directly from conditioned culture medium of transfected HEK-293T cells. This is a validated approach that has been used previously.^{(14, 32)} Protein expression was quantified by Western blot using an anti-FLAG tag antibody (FIG. 2, Panel A). Several variants (R233A, R233E, H241T, T175L, T175F, T175D, R206E and R206M) expressed well, others (D132A, D205Y, D132Y, T175Y and D132F) displayed reduced expression levels, and some (D132T, D173R, V174R and A232L) were not secreted at all. Some of these non-secreted mutants were detected in cell lysates, suggesting folding and/or secretion problems, while others were not expressed at all, possibly as a result of mRNA instability or another problem (FIG. 8). In addition, some selectivity-conferring mutations were combined into double mutant variants; however, these mutants either failed to express (D132Y-T175Y) or did not show any binding toward both BCMA and TACI (T175D-D205Y/K, T175D-R206E) (data not shown).

### 3. Determination of Receptor Binding by ELISA

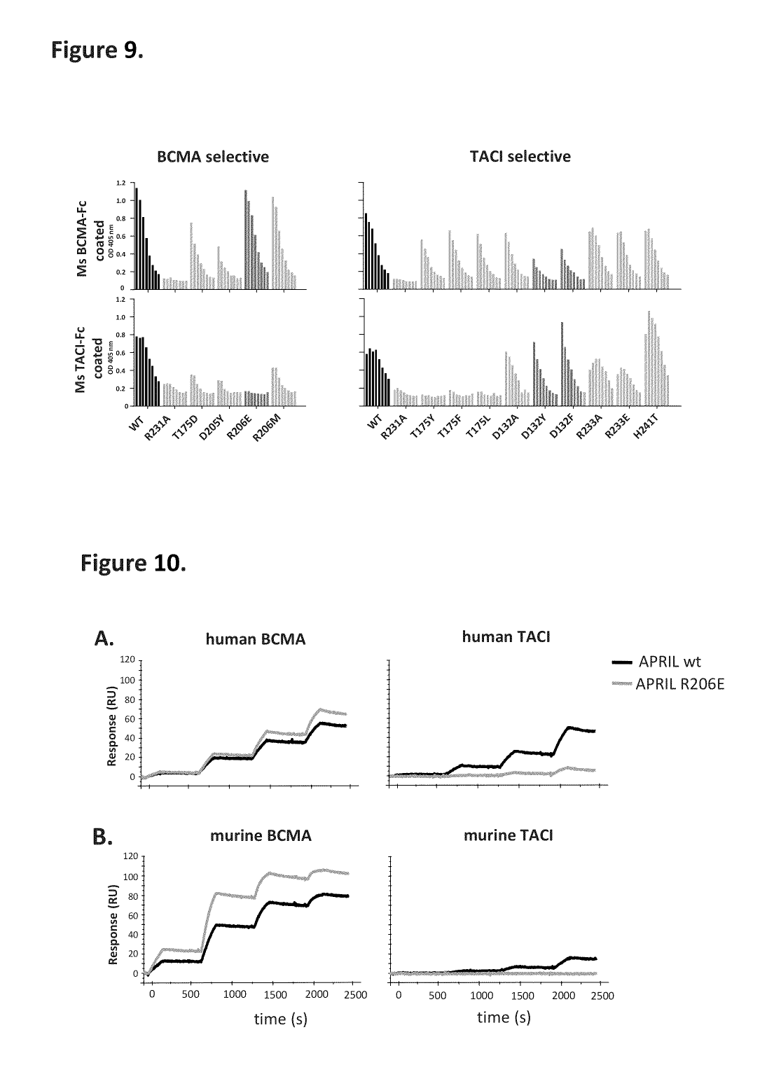

APRIL variants were tested using a receptor-binding ELISA as an initial screening assay to examine the real in vitro TACI and BCMA receptor binding and to determine receptor selectivity. Since we were interested in the relative changes in affinity of the APRIL variants for the TACI and BCMA receptors, we did not correct at this point for the different expression levels of the selected mutants. Thus, in the following experiments, although binding to the target receptor could seem weaker than for the WT variant, this is not necessarily the case (see below). Variants were grouped according to their predicted binding properties (i.e., being either TACI- or BCMA-specific, FIG. 1, Panel E.). R231A, previously shown by us to be a mutation that leads to loss of both TACI and BCMA binding, but not binding to HSPGs, was included as a negative control.^{(14, 32)} Binding to human BCMA-Fc was retained by all variants predicted to selectively bind BCMA (FIG. 2, Panel B). Although T175D was well expressed, it showed relatively lower binding to BCMA when compared to WT-APRIL; D205Y also showed decreased binding to BCMA. However, R206M and R206E retained a binding profile toward BCMA comparable to WT-APRIL. In contrast, when tested for binding toward human TACI-Fc, all these single variants showed significantly reduced binding compared to WT-APRIL. Therefore, all variants predicted to selectively bind BCMA indeed showed enhanced selectivity toward BCMA. However, binding of R206E to TACI-Fc was completely lost, indicating not only enhanced BCMA selectivity, but complete specificity. All APRIL mutants designed for being TACI-selective, retained binding to both BCMA and TACI, yet showed a preferential binding for TACI at the expense of BCMA (FIG. 2, Panel C). Mutants with the best TACI to BCMA binding ratio were D132Y and D132F. Although both the TACI or BCMA variants were not specifically designed to bind murine receptors, the ligands were also tested for their binding to the homologous mouse receptors that share ˜70% sequence identity with their human counterparts (FIG. 7, Panel B). Although one variant, H241T, showed an improved selectivity toward the mTACI, R206E, D132F and D132Y showed similar binding toward mBCMA-Fc and mTACI-Fc as that observed with human receptors (mBCMA-Fc and mTACI-Fc FIG. 9). The binding of APRIL to TACI and BCMA was quantified by surface plasmon resonance (SPR) (FIG. 10 and Tables 1 and 2). WT APRIL bound TACI- and BCMA-Fc, both human and mouse, with affinities comparable to previously published values (Tables 1 and 2).^{(9, 11, 12, 34)} The BCMA-specific mutant R206E bound both human and mouse BCMA with affinities comparable to WT-APRIL, while its binding to TACI was obviously reduced. Comparison of the affinities of R206E for BCMA and TACI shows that this variant is 25-fold more selective for BCMA than for TACI. Unfortunately, the TACI-selective variants D132Y and D132F could not be produced in sufficient quantities to obtain reliable SPR readings. Taken together, these initial screening assays highlight one variant, R206E, with specificity for BCMA, and two variants, D132F and D132Y, with selectivity towards TACI. All of these mutants bind similarly to human and mouse receptors.

### 4. R206E Shows Specificity for BCMA, while D132F and D132Y Show Selectivity Toward TACI

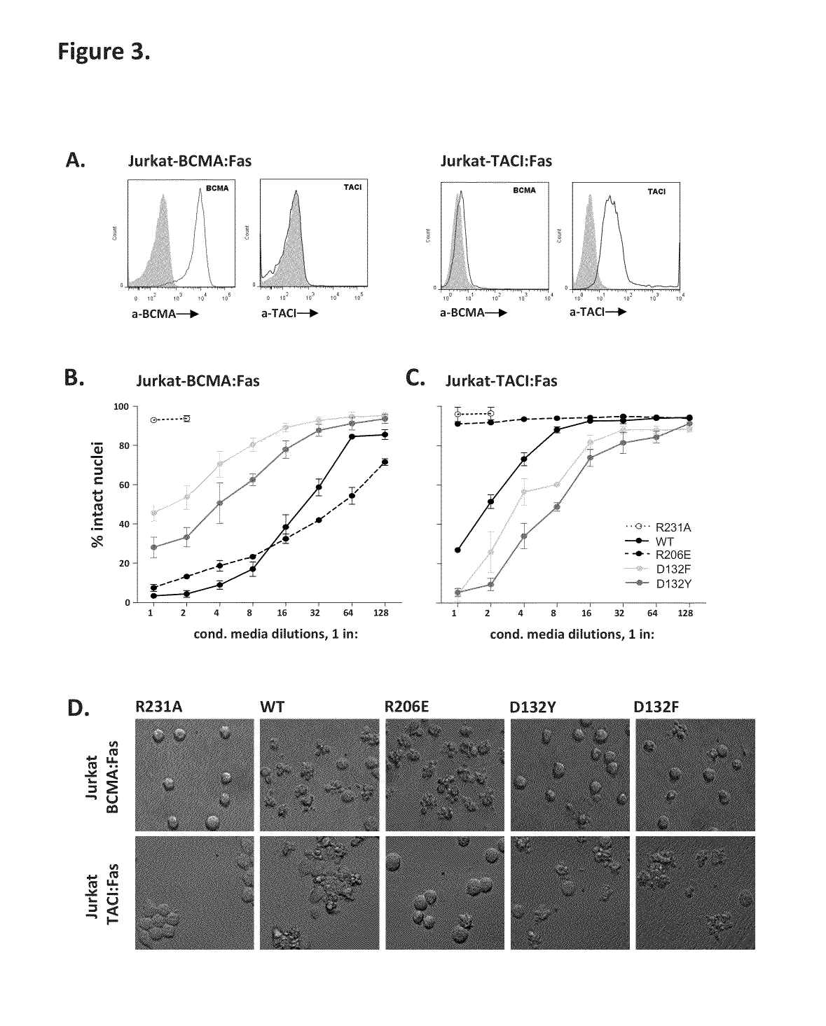

In order to study receptor selectivity of APRIL variants in a standardized cell-based assay, BCMA:Fas- and TACI:Fas-expressing Jurkat cells were used as a reporter system.⁽²⁸⁾ In this assay, binding of APRIL to chimeric receptors triggers the pro-apoptotic Fas signaling pathway, leading to cell death. Reporter cells indeed expressed their respective chimeric receptors on the surface, as shown by FACS staining (FIG. 3, Panel A). Both WT APRIL and R206E efficiently killed BCMA:Fas-expressing cells (FIG. 3, Panel B), but only WT APRIL, and not R206E, killed TACI:Fas reporter cells (FIG. 3, Panel C). Conversely, D132F and D132Y showed reduced activity on BCMA:Fas Jurkat cells (FIG. 3, Panel B), but enhanced activity on TACI:Fas Jurkat cells when compared to WT APRIL (FIG. 3, Panel C). Cell death was also evident at the morphological level, with numerous apoptotic blebs forming as early as one hour post-treatment initiation (FIG. 3, Panel D). Thus, the receptor specificity of R206E, and selectivity of D132F and D132Y were confirmed in a cell-based assay.

### 5. D132F and D132Y, but not R206E, Triggered TACI Internalization on Endogenously Expressed Receptors

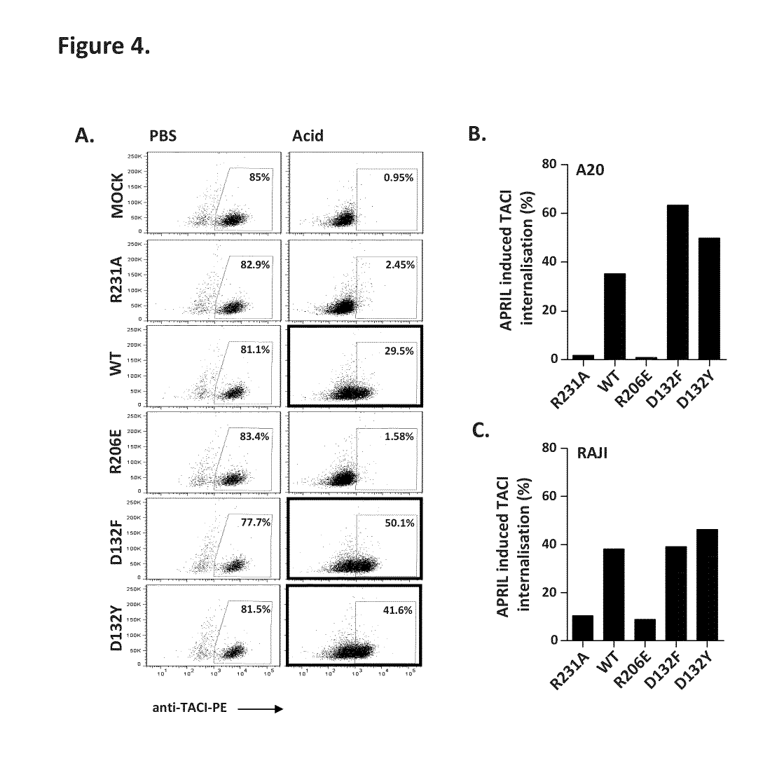

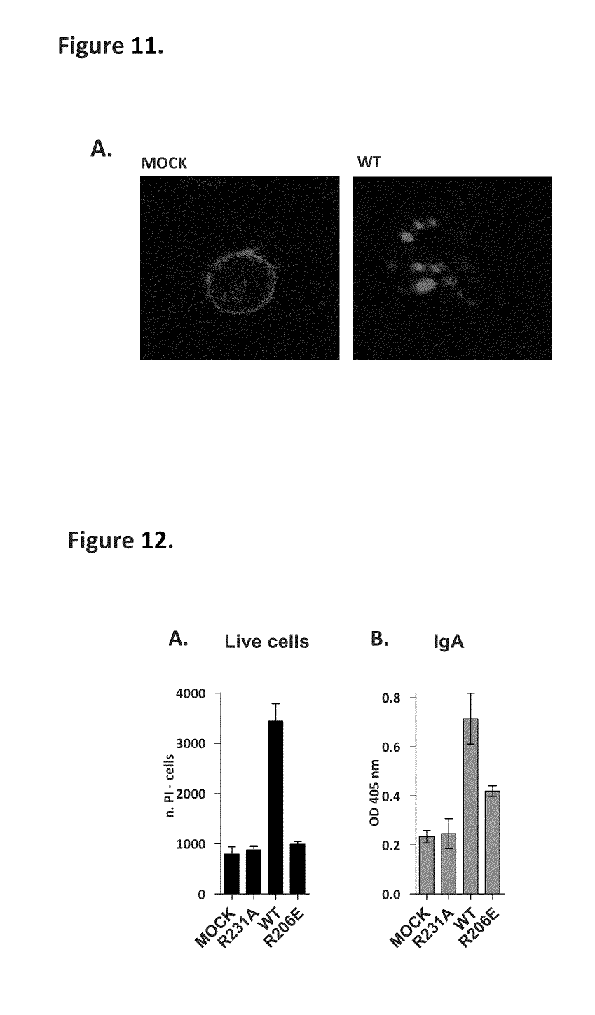

In order to test whether the R206E variant would also be unable to stimulate endogenous WT TACI, we used a receptor internalization assay.⁽³²⁾ We chose the mouse A20 cell line that has been shown to express high amounts of TACI.^{(10, 32)} Treatment with WT-APRIL for 90 minutes at 37° C. triggered TACI internalization, as shown by the high-PE signal retained after acid treatment, which marks the antibody that was internalized, together with the receptor (FIG. 4, Panel A, row 3, marked box). Visualization of stimulated cells by confocal microscopy confirmed in a more direct way that TACI was internalized (FIG. 11). The two TACI-selective ligands, D132F and D132Y, efficiently triggered TACI internalization at levels even higher than those achieved with WT APRIL (FIG. 4, Panel B, rows 5 and 6, marked boxes), in accordance with the ELISA binding and Jurkat killing assays. In contrast, R206E failed to trigger TACI internalization and was comparable to the “receptor-dead” R231A variant or to the mock control, where acid treatment completely quenched the extracellular PE signal due to lack of TACI internalization (FIG. 4, Panels A and B). Similar results were obtained on the human lymphoma cell line Raji, for which we recently showed expression of both TACI and BCMA⁽³²⁾ (FIG. 4, Panel C). These results point to the inability of R206E to stimulate endogenous TACI.

### 6. Distinct Effects of APRIL Variants on B Splenocytes Survival and IgA Production

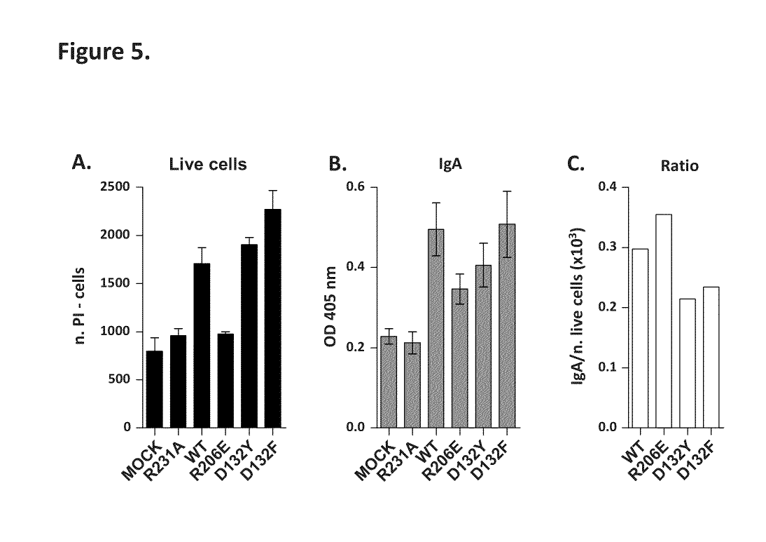

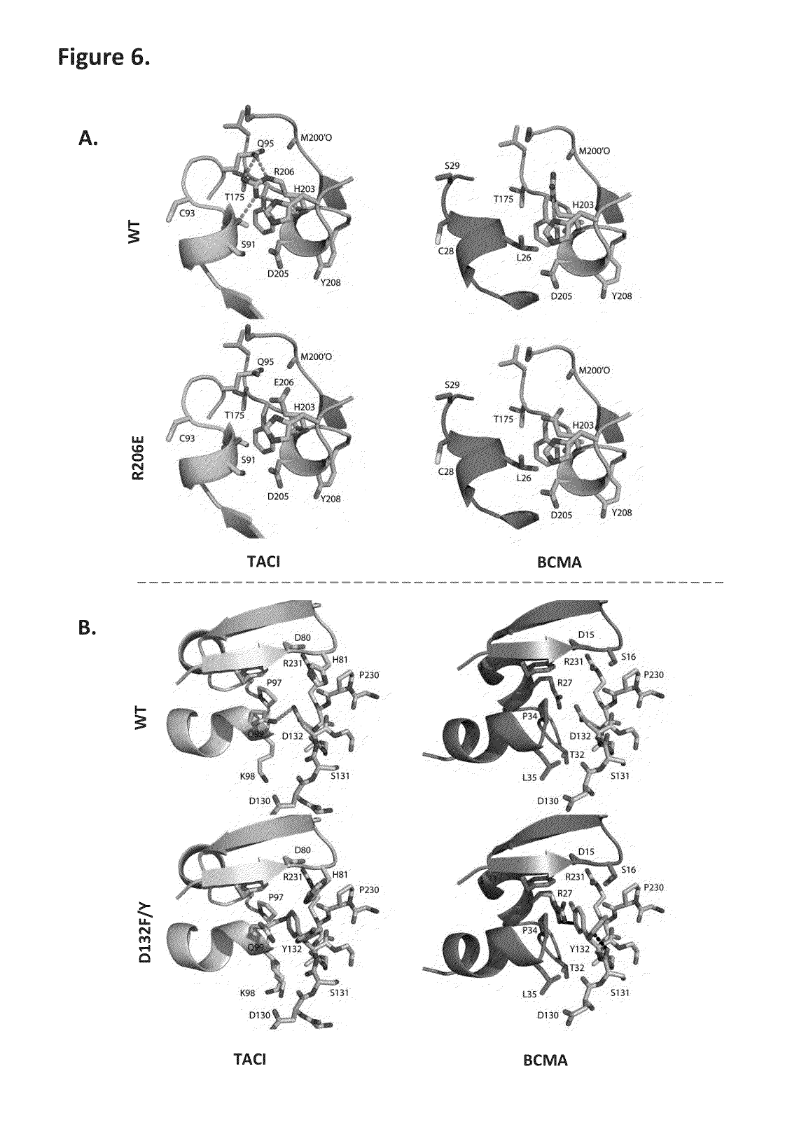

APRIL variants were tested for their activity on freshly isolated mouse B220⁺ splenocytes, which are known to respond to APRIL by increasing survival and IgA production.^{(14, 32)} WT APRIL increased the number of live murine B cells remaining after six days of culture by a factor of 2 (FIG. 5, Panel A) and also doubled the levels of IgA compared to the control (FIG. 5, Panel B). TACI-selective APRIL variants were slightly more potent than WT at increasing cell survival (FIG. 5, Panel A), although IgA production was not found to be proportionally increased (FIG. 5, Panels B and C). In contrast, the BCMA-specific variant R206E failed to increase live cell numbers, yet partially increased IgA levels in cell supernatants (FIG. 5, Panels A and B). This suggests that APRIL variants might prove useful at dissecting TACI- or BCMA-dependent B cell responses.

### Tables

TABLE 1
Affinities for Human and Mouse BCMA as measured by BIAcore.
The numbers represent the average of a global fit using three
curves from separate experiments.

| | | | |
|---|---|---|---|
| Protein | kₐ (M⁻¹s⁻¹) | k_{d} (s⁻¹) | K_{D} (M) |

Table 1a. Affinities for Human BCMA-Fc

| | | | |
|---|---|---|---|
| WT | 5.3 ± 0.1 10⁵ | 2.0 ± 0.1 10⁻⁴ | 3.8 ± 0.1 10⁻¹⁰ |
| R206E | 3.3 ± 0.0 10⁵ | 1.5 ± 0.0 10⁻⁴ | 4.6 ± 0.0 10⁻¹⁰ |

Table 1b. Affinities for Mouse BCMA-Fc

| | | | |
|---|---|---|---|
| WT | 8.9 ± 0.0 10⁵ | 1.3 ± 0.0 10⁻⁴ | 1.4 ± 0.1 10⁻¹⁰ |
| R206E | 16 ± 0.0 10⁵ | 1.5 ± 0.0 10⁻⁴ | 0.95 ± 0.3 10⁻¹⁰ |

TABLE 2
Affinities for Human and Mouse TACI as measured by BIAcore.

| | | | |
|---|---|---|---|
| Protein | kₐ (M⁻¹s⁻¹) | k_{d} (s⁻¹) | K_{D} (M) |

Table 2a. Affinities for Human TACI-Fc

| | | | |
|---|---|---|---|
| WT | 1.1 ± 0.1 10⁵ | 4.1 ± 0.1 10⁻⁴ | 39 ± 3 10⁻¹⁰ |
| R206E | 20 ± 29 10⁵ | 100 ± 90 10⁻⁴ | 110 ± 120 10⁻¹⁰ |
| R206M | 4.9 ± 0.0 10⁵ | 1.6 ± 0.0 10⁻⁴ | 3.2 ± 0.0 10⁻¹⁰ |

Table 2b. Affinities for Mouse TACI-Fc

| | | | |
|---|---|---|---|
| WT | 1.7 ± 0.1 10⁵ | 7.5 ± 0.1 10⁻⁴ | 44 ± 1 10⁻¹⁰ |
| R206E | | | RU too low, no fit |

TABLE 3
Primers for multi-mutagenesis. All primers were designed based on
the human APRIL DNA Genbank sequence record AF046888. Residue numbering
of the variants was based on the APRIL protein sequence as described in
the Expasy Uniprot record (O75888).

| | | | SEQ ID |
|---|---|---|---|
| Primer Name | Sequence (5′to 3′) | Tm | NO: |
| APRIL-D132A | tccaaggatgactccgctgtgacagaggtgatg | 79.14° C. | 15 |
| APRIL-D132F | acctccaaggatgactcctttgtgacagaggtgatgtg | 78.97° C. | 16 |
| APRIL-D132T | acctccaaggatgactccactgtgacagaggtgatgtg | 78.97° C. | 17 |
| APRIL-D173R | atagccaggtcctgtttcaacgcgtgactttcaccatgg | 79.04° C. | 18 |
| APRIL-V174R | ccaggtcctgtttcaagacaggactttcaccatgggtcag | 80.13° C. | 19 |
| APRIL-T175F | ggtcctgtttcaagacgtgtttttcaccatgggtcaggtg | 80.13° C. | 20 |
| APRIL-T175L | ggtcctgtttcaagacgtgctattcaccatgggtcaggtgg | 78.72° C. | 21 |
| APRIL-R233A | gtgtcataattccccgggcagcggcgaaacttaacc | 78.83° C. | 22 |
| APRIL-R233E | gagtgtcataattccccgggcagaggcgaaacttaacctc | 80.13° C. | 23 |
| APRIL-H241T | cgaaacttaacctctctccaactggaaccttcctgggg | 78.97° C. | 24 |
| APRIL-D132Y | cctccaaggatgactcctatgtgacagaggtgatg | 80.44° C. | 25 |
| APRIL- T175Y | ggtcctgtttcaagacgtgtatttcaccatgggtcaggtg | 80.13° C. | 26 |
| APRIL- T192R | ggccaaggaaggcaggagaggctattccgatgtataagaa | 79.10° C. | 27 |
| APRIL- A232L | gagtgtcataattccccggttaagggcgaaacttaacctc | 79.10° C. | 28 |
| APRIL- T175D | ggtcctgtttcaagacgtggatttcaccatgggtcaggtg | 79.10° C. | 29 |
| APRIL- R206E | ctcccacccggacgaggcctacaacagc | 78.07° C. | 30 |
| APRIL- D205K | gccctcccacccgaagcgggcctacaaca | 79.60° C. | 31 |
| APRIL- D205Y | gccctcccacccgtatcgggcctacaaca | 78.19° C. | 32 |
| APRIL- R206M | cctcccacccggacatggcctacaacagct | 78.30° C. | 33 |

### Materials and Methods

### 1. Computational Design of Selective Variants

X-ray crystal structures of the extracellular domain (ECD) of murine APRIL in complex with the ECD of human TACI (PDB 1xu1) and the ECD of human BCMA (PDB 1xu2) have been solved at a resolution of 1.9 Å and 2.35 Å, respectively.⁽²¹⁾ Computational design of receptor selective mutants was performed as described previously.^{(22, 23, 26)} In short, amino acid residues with Van der Waals clashes, bad torsion angles or with a high energy in the crystal structure, were repaired by replacing the side chain conformations (rotamers) observed in the x-ray structure by lower energy rotamers; hydrogen bond networks were optimized using the Repair PDB option of the FoldX protein design algorithm.^{(19, 27)} Next, a model of human APRIL in complex with human TACI and human BCMA was constructed by FoldX in silico mutagenesis of each of the non-conserved murine residues to its homologous human counterpart. Each residue in the receptor binding interface of human APRIL was subsequently mutated by FoldX to all other 19 naturally occurring amino acids using the BuildModel function. The effect on the interaction energy with TACI and BCMA was calculated as the difference in interaction energy (ΔΔG; in kcal/mol) between the interaction energy of the mutant and the wild-type amino acid, using the AnalyseComplex option. Amino acid substitutions were selected that: 1) caused a decrease in interaction energy toward one of the receptors or 2) caused an increase in interaction energy toward one receptor, while causing either a decrease in interaction energy or showing a neutral effect toward the other receptor or 3) caused an increase in interaction energy for both receptors but showing a different magnitude in change for one receptor over the other. Because some variants (mainly from the D132 series) showed severe intra-chain Van der Waals clashes upon mutation, it was decided to report the average interaction energy corrected for intra-chain Van der Waals clashes. This corrected interaction energy was calculated by summing the average interaction energy and the average ΔΔGintraclash energy, where ΔΔGintraclash is the difference in intra-chain clash energy (ΔΔGintraclash; in kcal/mol) between the intra-chain clash energy of the mutant and the wild-type amino acid. The reported corrected interaction energy was capped at +4 kcal/mol.

### 2. Generation of Variants

Flag-tagged APRIL variants selective for a receptor were generated using a Quick Change site-directed mutagenesis kit (Agilent Technologies, Santa Clara, USA) according to the manufacturer's guidelines, using the primers listed in Table 3. A pcDNA3.1 construct containing FLAG-tagged wild-type soluble APRIL (amino acid 105 to 250, numbering according to UniprotKB/Swiss-Prot 075888) was used as the PCR template and was described previously.⁽¹⁴⁾ Plasmid DNA of clones was isolated (BIOKÉ, Leiden, The Netherlands) and presence of the mutation(s) was verified by DNA sequencing. Positive clones were selected and grown up for large-scale plasmid DNA isolation and used for subsequent transfections.

### 3. Cell Culture

Human Jurkat and Raji cells and mouse A20 cells were cultured in RPMI-1640 (Gibco Life Technologies, Breda, NL). Primary mouse B cells were cultured in RPMI-1640 supplemented with 50 μM of 2-mercaptoethanol. 293T cells were cultured in Iscove's Modified Dulbecco Media (IMDM). All media were supplemented with 8% FCS, 2 mM L-glutamine, 40 μg/m1 penicillin and 40 U/ml streptomycin and maintained at 37° C. with 5% CO₂.

### 4. Expression of APRIL Variants

To test the expression of the individual Flag-tagged APRIL variants, 293T cells were grown to 60% confluence in a six-well plate and transfected with the different variants using calcium phosphate precipitation. Following transfection, the cells were kept in culture for 72 hours before supernatant containing the soluble mutants was harvested and stored at −20° C. Expression of each of the APRIL mutants was then tested by Western blotting and the relative concentrations assessed. Briefly, supernatants were resolved on a 15% polyacrylamide gel, transferred to a nitrocellulose membrane and blocked overnight using Odyssey blocking buffer (LiCor Biosciences, Cambridge, United Kingdom) diluted 1:1 with PBS. The membrane was then incubated with mouse anti-FLAG-M2 (Sigma-Aldrich, Zwijndrecht, The Netherlands) at a concentration of 1 in 5000 diluted in PBS/0.2% TWEEN®20; the secondary antibody used was IRD800-coupled anti-mouse IgG1 (Westburg, Leusden, The Netherlands) diluted in PBS/0.2% TWEEN®20 and 0.02% SDS. Blots were visualized using a near-infrared imaging system (Odyssey, LiCor) according to the manufacturer's instructions, which allows quantification of bands to give a relative estimate of protein concentration.

### 5. Receptor Binding ELISA

The relative binding of the variants to either BCMA or TACI, was tested using a binding ELISA. The following proteins were used: human BCMA-Fc, human TACI-Fc (both generated in-house), mouse BCMA-Fc (R&D Systems, Abingdon, UK), mouse TACI-Fc (R&D Systems); in all cases the Fc portion is from human IgG1. BCMA-Fc and TACI-Fc were coated on 96-well Nunc Maxisorp plates (Thermo Fischer Scientific, Roskilde, Denmark) at concentrations of 1 μg/ml and 2 μg/ml, respectively, in 0.5 M sodium bicarbonate buffer pH 9.5, overnight at 4° C. Plates were then blocked with 5% BSA for one hour at 37° C. Soluble APRIL variants in the form of tissue culture conditioned medium were then added to the plate for two hours at 37° C. Following APRIL binding, plates were washed three times with PBS, 0.05% TWEEN®20. Bound APRIL was detected with 1 μg/ml of HRP-coupled anti-FLAG M2 and visualized with 2,2′-azino-bis(3-ethylbenzothiazoline-6-sulphonic acid (ABTS) (Sigma-Aldrich). The absorbance was read at 405 nm using a UV-VIS microplate reader (BioRad, Veenendaal, The Netherlands).

### 6. Surface Plasmon Resonance

In order to further assess the binding properties of the mutants and to measure apparent affinities and kinetics of receptor binding, a Surface Plasmon Resonance based receptor binding assay was performed on a Biacore 2000 (GE Healthcare, Diegem, Belgium). Anti-FLAG-M2 monoclonal antibody (Sigma-Aldrich) was diluted at a concentration of 5 μg/ml in 10 mM sodium acetate pH 4.5 and covalently immobilized (approx 1500 RU) to a CM-5 sensor chip (GE Healthcare), using standard amine coupling chemistry according to manufacturer's guidelines (Amine coupling kit, GE Healthcare). The different FLAG-APRIL variants in the form of tissue culture supernatants were captured onto the chip via the FLAG-tag at 10 μl/minute for five minutes, giving capture levels ranging from 159 to 207 RU. As a reference lane, one of the flow cells was left free of soluble FLAG-APRIL, to control for any background binding. BCMA- or TACI-Fc were then injected for three minutes over all four flow cells at 30 μl/minutes at increasing concentrations (ranging from 1-50 nM) using the single cycle kinetics method at 25° C. Dissociation was monitored for five minutes. The resulting curves were fitted using a 1:1 Langmuir model using BIAevaluation software 4.1. For each combination of FLAG-APRIL and BCMA- or TACI-Fc, the apparent kₐ, k_{d} and K_{D} were calculated from a global fit of binding curves from at least three separate experiments. Non-specific binding in the reference cell was subtracted before curve fitting. Regeneration of the anti-FLAG antibody surface was carried out with a 10-minute injection at 30 μl/minute of a mixture of ⅓ volume of TBS pH 11.5, ⅓ volume of Ionic solution and ⅓ volume of water. The Ionic solution was comprised of KSCN (0.46 M), MgCl₂ (1.83 M), urea (0.92 M) and guanidine-HCl (1.83 M).

### 7. Generation of Jurkat-BCMA:Fas and TACI:Fas Reporter Cells, and Killing Assay

Jurkat-BCMA:Fas-2309 cl13 reporter cells were generated as described.⁽²⁸⁾ TACI:Fas Jurkat cells reporter cell lines were generated essentially as described previously for EDAR:Fas cells.⁽²⁹⁾ 293T cells were transiently transfected with pMSCVpuro-TACI:Fas and co-transfected with the pHIT60 and VSV-G plasmids, containing the sequences for gag-pol and VSV-G, respectively. pMSCVpuro-TACI:Fas encodes the haemaglutinin signal peptide (amino acid sequence MAIIYLILLFTAVRG), part of the extracellular domain of human TACI (amino acids 2-118), amino acids VD and the transmembrane and intracellular domains of human Fas (amino acids 169-335). After transfection, 293T cells were incubated for 48 hours in RPMI supplemented with 10% FCS. Six ml of virus-containing 293T cell supernatants supplemented with 8 μg/ml of polybrene were added to 10⁶ Fas-deficient Jurkat-JOM2 cells (a kind gift of Olivier Micheau, University of Dijon, France) in two times 3 ml additions for 3 and 16 hours, respectively, after which time, cells were cultured in RPMI 10% for 72 hours and then selected with 0.5 μg/ml of puromycin and cloned. Clones were screened for their selective sensitivity to Fc-BAFF but not Fc-EDA1⁽³⁰⁾ and one clone (clone 112) was selected for further experimentation. For the killing assay, 3×10⁴ Jurkat cells were seeded per well in a 96-well plate and stimulated with doubling dilutions of quantity-matched supernatants for a period of 16 hours. Cells were subsequently harvested, spun down and re-suspended in 250 μl of Nicoletti Buffer containing 50 μg/ml of propidium iodide and stored for at least 24 hours at 4° C. (as described in reference (31)). Analysis of apoptosis was assessed by flow cytometric measurement of PI stained nuclei using a FACScalibur system (Becton Dickinson, San Jose, Calif., USA).

### 8. Internalization Assay

To determine receptor internalization, 5×10⁵ cells were labeled with phycoerythrin (PE)-conjugated anti-human or anti-mouse TACI antibodies (Clones: 1A1-K21-M22 and 8F10, respectively, BD Biosciences, Breda, The Netherlands) and incubated with conditioned medium containing matched amounts of APRIL variants for a period of 90 minutes at 37° C. (the optimal time point was determined in a time-course experiment). Following incubation, cells were cooled on ice to halt endocytosis, treated for one minute with either acid solution (0.154 M NaCl pH 2, to strip off surface-exposed antibody) or PBS, 1% BSA as a control and then analyzed by FACS for the presence of the remaining PE-label. The efficacy of the acid stripping was tested and optimized previously.⁽³²⁾ All APRIL receptor staining on lymphoma cells (mouse and human) were performed after incubation with FcR blocking reagent (Miltenyi Biotech, Leiden, The Netherlands). TACI internalization was also studied using confocal microscopy. Cells were stained with PE-conjugated anti-mouse TACI antibody, incubated with APRIL in conditioned medium (as described above for FACS analysis), re-suspended in mounting medium (VectaShield, Brunschwig Chemie, Amsterdam, NL) and transferred onto a glass slide. Cells were kept on ice before microscopy.

### 9. B Cell Assay

B cells were purified from murine splenocytes using magnetic activated cell separation (MACS) with CD45R/B220 MACS beads (Miltenyi Biotec, Utrecht, The Netherlands). Purified B cells were then seeded in 96-well round-bottomed microtiter plates at a density of 2×10⁵ cells/well, and incubated with diluted conditioned media containing the APRIL variants. After six days of incubation, viability was assessed using PI exclusion and supernatants were assayed for IgA by ELISA. Coated 96-well plates (2 μg/ml anti-mouse Ig, Southern Biotech) were blocked with PBS, 5% BSA and, following washes with PBS, 0.05% TWEEN®20, incubated at 37° C. for one hour with the collected supernatants. Bound IgA were detected with HRP-labeled anti-mouse-IgA (Southern Biotech) and ABTS (Sigma-Aldrich).

### REFERENCE LIST

1. Hahne M., T. Kataoka, M. Schroter, K. Hofmann, M. Irmler, J. L. Bodmer, P. Schneider, T. Bornand, N. Holler, L. E. French, B. Sordat, D. Rimoldi, and J. Tschopp (1998). APRIL, a new ligand of the tumor necrosis factor family, stimulates tumor cell growth. J. Exp. Med. 188:1185-1190
2. Castigli E., S. A. Wilson, S. Scott, F. Dedeoglu, S. Xu, K. P. Lam, R. J. Bram, H. Jabara, and R. S. Geha (2005). TACI and BAFF-R mediate isotype switching in B cells. J. Exp. Med. 201:35-39
3. Castigli E., S. Scott, F. Dedeoglu, P. Bryce, H. Jabara, A. K. Bhan, E. Mizoguchi, and R. S. Geha (2004). Impaired IgA class switching in APRIL-deficient mice. Proc. Natl. Acad. Sci. U.S.A 101:3903-3908
4. O′Connor B. P., V. S. Raman, L. D. Erickson, W. J. Cook, L. K. Weaver, C. Ahonen, L. L. Lin, G. T. Mantchev, R. J. Bram, and R. J. Noelle (2004). BCMA is essential for the survival of long-lived bone marrow plasma cells. J. Exp. Med. 199:91-98
5. Kimberley F. C., J. P. Medema, and M. Hahne (2009). APRIL in B-cell malignancies and autoimmunity. Results Probl. Cell Differ. 49:161-182
6. Deshayes F., G. Lapree, A. Portier, Y. Richard, P. Pencalet, D. Mahieu-Caputo, P. Horellou, and A. Tsapis (2004). Abnormal production of the TNF-homologue APRIL increases the proliferation of human malignant glioblastoma cell lines via a specific receptor. Oncogene 23:3005-3012
7. Mhawech-Fauceglia P., G. Kaya, G. Sauter, T. McKee, O. Donze, J. Schwaller, and B. Huard (2006). The source of APRIL up-regulation in human solid tumor lesions. J. Leukoc. Biol. 80:697-704
8. Lascano V., L. F. Zabalegui, K. Cameron, M. Guadagnoli, M. Jansen, M. Burggraaf, M. Versloot, H. Rodermond, C. van der Loos, C. E. Carvalho-Pinto, H. Kalthoff, J. P. Medema, and M. Hahne (2012). The TNF family member APRIL promotes colorectal tumorigenesis. Cell Death. Differ.
9. Marsters S. A., M. Yan, R. M. Pitti, P. E. Haas, V. M. Dixit, and A. Ashkenazi (2000). Interaction of the TNF homologues BLyS and APRIL with the TNF receptor homologues BCMA and TACI. Curr. Biol. 10:785-788
10. Rennert P., P. Schneider, T. G. Cachero, J. Thompson, L. Trabach, S. Hertig, N. Holler, F. Qian, C. Mullen, K. Strauch, J. L. Browning, C. Ambrose, and J. Tschopp (2000). A soluble form of B cell maturation antigen, a receptor for the tumor necrosis factor family member APRIL, inhibits tumor cell growth. J. Exp. Med. 192:1677-1684
11. Wu Y., D. Bressette, J. A. Carrell, T. Kaufman, P. Feng, K. Taylor, Y. Gan, Y. H. Cho, A. D. Garcia, E. Gollatz, D. Dimke, D. LaFleur, T. S. Migone, B. Nardelli, P. Wei, S. M. Ruben, S. J. Ullrich, H. S. Olsen, P. Kanakaraj, P. A. Moore, and K. P. Baker (2000). Tumor necrosis factor (TNF) receptor superfamily member TACI is a high affinity receptor for TNF family members APRIL and BLyS. J. Biol. Chem. 275:35478-35485
12. Yu G., T. Boone, J. Delaney, N. Hawkins, M. Kelley, M. Ramakrishnan, S. McCabe, W. R. Qiu, M. Kornuc, X. Z. Xia, J. Guo, M. Stolina, W. J. Boyle, I. Sarosi, H. Hsu, G. Senaldi, and L. E. Theill (2000). APRIL and TALL-I and receptors BCMA and TACI: system for regulating humoral immunity. Nat. Immunol. 1:252-256
13. Ingold K., A. Zumsteg, A. Tardivel, B. Huard, Q. G. Steiner, T. G. Cachero, F. Qiang, L. Gorelik, S. L. Kalled, H. Acha-Orbea, P. D. Rennert, J. Tschopp, and P. Schneider (2005). Identification of proteoglycans as the APRIL-specific binding partners. J. Exp. Med. 201:1375-1383
14. Kimberley F. C., L. van Bostelen, K. Cameron, G. Hardenberg, J. A. Marquart, M. Hahne, and J. P. Medema (2009). The proteoglycan (heparan sulfate proteoglycan) binding domain of APRIL serves as a platform for ligand multimerization and cross-linking FASEB J. 23:1584-1595
15. Bischof D., S. F. Elsawa, G. Mantchev, J. Yoon, G. E. Michels, A. Nilson, S. L. Sutor, J. L. Platt, S. M. Ansell, B. G. von, and R. J. Bram (2006). Selective activation of TACI by syndecan-2. Blood 107:3235-3242
16. Hatzoglou A., J. Roussel, M. F. Bourgeade, E. Rogier, C. Madry, J. Inoue, O. Devergne, and A. Tsapis (2000). TNF receptor family member BCMA (B cell maturation) associates with TNF receptor-associated factor (TRAF) 1, TRAF2, and TRAF3 and activates NF-kappa B, elk-1, c-Jun N-terminal kinase, and p38 mitogen-activated protein kinase. J. Immunol. 165:1322-1330
17. Xia X. Z., J. Treanor, G. Senaldi, S. D. Khare, T. Boone, M. Kelley, L. E. Theill, A. Colombero, I. Solovyev, F. Lee, S. McCabe, R. Elliott, K. Miner, N. Hawkins, J. Guo, M. Stolina, G. Yu, J. Wang, J. Delaney, S. Y. Meng, W. J. Boyle, and H. Hsu (2000). TACI is a TRAF-interacting receptor for TALL-1, a tumor necrosis factor family member involved in B cell regulation. J. Exp. Med. 192:137-143
18. Kanno Y., D. Sakurai, H. Hase, H. Kojima, and T. Kobata (2010). TACI induces cIAP 1-mediated ubiquitination of NIK by TRAF2 and TANK to limit non-canonical NF-kappaB signaling. J. Recept. Signal. Transduct. Res. 30:121-132
19. Guerois R., J. E. Nielsen, and L. Serrano (2002). Predicting changes in the stability of proteins and protein complexes: a study of more than 1000 mutations. J. Mol. Biol. 320:369-387
20. Schymkowitz J. W., F. Rousseau, I. C. Martins, J. Ferkinghoff-Borg, F. Stricher, and L. Serrano (2005). Prediction of water and metal binding sites and their affinities by using the Fold-X force field. Proc. Natl. Acad. Sci. U.S.A. 102:10147-10152
21. Hymowitz S. G., D. R. Patel, H. J. Wallweber, S. Runyon, M. Yan, J. Yin, S. K. Shriver, N. C. Gordon, B. Pan, N. J. Skelton, R. F. Kelley, and M. A. Starovasnik (2005). Structures of APRIL-receptor complexes: like BCMA, TACI employs only a single cysteine-rich domain for high affinity ligand binding. J. Biol. Chem. 280:7218-7227
22. Reis C. R., A. M. van der Sloot, A. Natoni, E. Szegezdi, R. Setroikromo, M. Meijer, K. Sjollema, F. Stricher, R. H. Cool, A. Samali, L. Serrano, and W. J. Quax (2010). Rapid and efficient cancer cell killing mediated by high-affinity death receptor homotrimerizing TRAIL variants. Cell Death. Dis. 1, e83
23. van der Sloot A. M., V. Tur, E. Szegezdi, M. M. Mullally, R. H. Cool, A. Samali, L. Serrano, and W. J. Quax. (2006). Designed tumor necrosis factor-related apoptosis-inducing ligand variants initiating apoptosis exclusively via the DR5 receptor. Proc. Natl. Acad. Sci. U.S.A. 103:8634-8639
24. van der Sloot A. M. and W. J. Quax (2011). Computational design of TNF ligand-based protein therapeutics. Adv. Exp. Med. Biol. 691:521-534
25. van der Sloot A. M., C. Kiel, L. Serrano, and F. Stricher (2009). Protein design in biological networks: from manipulating the input to modifying the output. Protein Eng. Des. Sel. 22:537-542
26. Tur V., A. M. van der Sloot, C. R. Reis, E. Szegezdi, R. H. Cool, A. Samali, L. Serrano, and W. J. Quax (2008). DR4-selective tumor necrosis factor-related apoptosis-inducing ligand (TRAIL) variants obtained by structure-based design. J. Biol. Chem. 283:20560-20568
27. Schymkowitz J., J. Borg, F. Stricher, R. Nys, F. Rousseau, and L. Serrano (2005). The FoldX web server: an online force field. Nucleic Acids Res. 33:W382-W388
28. Bossen C., T. G. Cachero, A. Tardivel, K. Ingold, L. Willen, M. Dobles, M. L. Scott, A. Maquelin, E. Belnoue, C. A. Siegrist, S. Chevrier, H. Acha-Orbea, H. Leung, F. Mackay, J. Tschopp, and P. Schneider (2008). TACI, unlike BAFF-R, is solely activated by oligomeric BAFF and APRIL to support survival of activated B cells and plasmablasts. Blood 111:1004-1012
29. Swee L. K., K. Ingold-Salamin, A. Tardivel, L. Willen, O. Gaide, M. Favre, S. Demotz, M. Mikkola, and P. Schneider (2009). Biological activity of ectodysplasin A is conditioned by its collagen and heparan sulfate proteoglycan-binding domains. J. Biol. Chem. 284:27567-27576
30. Bossen C., K. Ingold, A. Tardivel, J. L. Bodmer, O. Gaide, S. Hertig, C. Ambrose, J. Tschopp, and P. Schneider (2006). Interactions of tumor necrosis factor (TNF) and TNF receptor family members in the mouse and human. J. Biol. Chem. 281:13964-13971
31. Mullauer F. B., J. H. Kessler, and J. P. Medema (2009). Betulinic acid induces cytochrome c release and apoptosis in a Bax/Bak-independent, permeability transition pore dependent fashion. Apoptosis. 14:191-202
32. Guadagnoli M., F. C. Kimberley, U. Phan, K. Cameron, P. M. Vink, H. Rodermond, E. Eldering, A. P. Kater, E. H. van, and J. P. Medema (2011). Development and characterization of APRIL antagonistic monoclonal antibodies for treatment of B-cell lymphomas. Blood 117:6856-6865
33. Kim H. M., K. S. Yu, M. E. Lee, D. R. Shin, Y. S. Kim, S. G. Paik, O. J. Yoo, H. Lee, and J. O. Lee (2003). Crystal structure of the BAFF-BAFF-R complex and its implications for receptor activation. Nat. Struct. Biol. 10:342-348
34. Day E. S., T. G. Cachero, F. Qian, Y. Sun, D. Wen, M. Pelletier, Y. M. Hsu, and A. Whitty (2005). Selectivity of BAFF/BLyS and APRIL for binding to the TNF family receptors BAFFR/BR3 and BCMA. Biochemistry 44:1919-1931
35. He B., R. Santamaria, W. Xu, M. Cols, K. Chen, I. Puga, M. Shan, H. Xiong, J. B. Bussel, A. Chiu, A. Puel, J. Reichenbach, L. Marodi, R. Doffinger, J. Vasconcelos, A. Issekutz, J. Krause, G. Davies, X. Li, B. Grimbacher, A. Plebani, E. Meffre, C. Picard, C. Cunningham-Rundles, J. L. Casanova, and A. Cerutti (2010). The transmembrane activator TACI triggers immunoglobulin class switching by activating B cells through the adaptor MyD88. Nat. Immunol. 11:836-845
36. Mackay F., P. Schneider, P. Rennert, and J. Browning (2003). BAFF AND APRIL: a tutorial on B cell survival. Annu. Rev. Immunol. 21:231-264
37. Cancro M. P. (2008). Living in context with the survival factor BAFF. Immunity. 28:300-301
38. Gardam S., F. Sierro, A. Basten, F. Mackay, and R. Brink (2008). TRAF2 and TRAF3 signal adapters act cooperatively to control the maturation and survival signals delivered to B cells by the BAFF receptor. Immunity. 28:391-401
39. López-Fraga M., R. Fernandez, J. P. Albar, M. Hahne (2001). Biologically active APRIL is secreted following intracellular processing in the Golgi apparatus by furin convertase. EMBO Rep. 2(10):945-51. Epub 2001 Sep. 24
40. Pradet-Balade B., J. P. Medema, M. Lopez-Fraga, J. C. Lozano, G. M. Kolfschoten, A. Picard, C. Martínez-A, J. A. Garcia-Sanz, M. Hahne (2002). An endogenous hybrid mRNA encodes TWE-PRIL, a functional cell surface TWEAK-APRIL fusion protein. EMBO J. 21(21):5711-20.
41. Kolfschoten G. M., B. Pradet-Balade, M. Hahne, and J. P. Medema (2003). TWE-PRIL; a fusion protein of TWEAK and APRIL. Biochem. Pharmacol. 66(8):1427-32.
42. Kimberley F. C., M. Hahnem, and J. P. Medemam (2009). APRIL hath put a spring of youth in everything: Relevance of APRIL for survival. J. Cell Physiol. 218(1):1-8. doi: 10.1002/jcp.21561.

What is claimed is:

1. A peptide comprising an amino acid sequence selected from the group consisting of SEQ ID NO:2, SEQ ID NO:4, SEQ ID NO:6, SEQ ID NO:8, SEQ ID NO:10, SEQ ID NO:12, and a fragment thereof corresponding to amino acid residues 104-250 or amino acid residues 105-250 of SEQ ID NO:2, SEQ ID NO:4, SEQ ID NO:6, SEQ ID NO:8, SEQ ID NO:10, or SEQ ID NO:12.

2. A recombinant polynucleotide encoding the peptide of claim 1.

3. An expression vector comprising the recombinant polynucleotide of claim 2.

4. A host cell comprising the recombinant polynucleotide of claim 2.

5. A host cell comprising the expression vector of claim 3.

6. A method for producing a peptide, the method comprising:
culturing the host cell of claim 5 under conditions suitable for expression of the recombinant polynucleotide.

7. A method of treating a subject diagnosed as suffering from an APRIL-associated disorder, the method comprising:
administering the peptide of claim 1 to the subject so as to treat the APRIL-associated disorder.

8. A method of ex vivo manipulating a cell, the method comprising:
utilizing the peptide of claim 1 for the ex vivo manipulation of cells.

9. A method of ex vivo manipulating a cell, the method comprising:
utilizing the recombinant polynucleotide of claim 2 for the ex vivo manipulation of cells.

10. The peptide of claim 1, together with a pharmaceutically acceptable carrier.

11. A method of treating a subject diagnosed as suffering from an APRIL-associated disorder, the method comprising:
administering the peptide of claim 10 to the subject so as to treat the APRIL-associated disorder.

12. A peptide comprising amino acid residues 104-250 or amino acid residues 105-250 of SEQ ID NO:2, SEQ ID NO:4, SEQ ID NO:6, SEQ ID NO:8, SEQ ID NO:10, or SEQ ID NO:12.

13. The peptide of claim 12, wherein the peptide consists of amino acid residues 105-250 of SEQ ID NO:2, SEQ ID NO:4, SEQ ID NO:6, SEQ ID NO:8, SEQ ID NO:10, or SEQ ID NO:12.

14. The peptide of claim 12, wherein the peptide consists of amino acid residues 104-250 of SEQ ID NO:2, SEQ ID NO:4, SEQ ID NO:6, SEQ ID NO:8, SEQ ID NO:10, or SEQ ID NO:12.
